(19) United States
(12) Patent Application Publication
(10) Pub. No.: US 2006/0217340 A1
(43) Pub. Date: Sep. 28, 2006

(54) Methods and products for treating hypertension by modulation of TRPC3 channel activity

(75) Inventors: Joseph E. Braydon, South Burlington, VT (US); Brian Waldron, Apex, NC (US); Stacey Reading, Burlington, VT (US); Scott Earley, Fort Collins, CO (US)

Correspondence Address: Helen C. Lockhart, Ph.D.;Wolf, Greenfield & Sacks, P.C., 600 Atlantic Avenue, Boston, MA, 02210-2206, US

(73) Assignee: University of Vermont and State Agricultural College, Burlington, VT (US)

(21) Appl. No.: 11/388,194

(22) Filed: Mar. 23, 2006

Related U.S. Application Data
(60) Provisional application No. 60/664,511, filed on Mar. 23, 2005.
(60) Provisional application No. 60/665,238, filed on Mar. 24, 2005.

(51) Int. Cl. A61K 48/00; A61K 31/553; A61K 31/455

(52) U.S. Cl. 514/44; 514/355

(57) ABSTRACT

The invention relates to methods and products for treatment of hypertension, high blood pressure and vasospasm. Specifically, TRPC3 channel inhibitors and related compositions and kits are described.

### RELATED APPLICATIONS

This application claims priority under 35 U.S.C. § 119(e) to U.S. Provisional Application Ser. No. 60/664,511, entitled “MODULATION OF TRPC3 CHANNEL ACTIVITY AS A METHOD FOR TREATING HYPERTENSION”, filed on Mar. 23, 2005, and U.S. Provisional Application Ser. No. 60/665,238, entitled “METHODS AND PRODUCTS FOR TREATING HYPERTENSION BY MODULATION OF TRPC3 CHANNEL ACTIVITY”, filed on Mar. 24, 2005, which are herein incorporated by reference in their entirety.

### FEDERALLY SPONSORED RESEARCH

This invention was made with Government support under NIH grant number HL 58231. Accordingly, the Government may have certain rights in this invention.

### FIELD OF THE INVENTION

The present invention relates to TRPC3 channel inhibitors, compositions and kits thereof and methods for the use of TRPC3 channel inhibitors in the treatment of diseases such as hypertension, vasospasm and in methods for lowering blood pressure.

### BACKGROUND OF THE INVENTION

Arterial diameter is a primary effector of blood flow and pressure. Influx of extracellular Ca²⁺ through voltage dependent L-type Ca²⁺ channels located in the arterial smooth muscle cell (SMC) plasma membrane is central in the control of cerebrovascular arterial diameter (Nelson et al. 1990 Am J Physiol Heart Circ Physiol 259, C3-C 18). Membrane depolarization opens L-type Ca²⁺ channels and their steep voltage-dependence means that small changes in membrane potential dramatically affect channel open probability, Ca²⁺ influx, and vascular tone. Various agonists that bind to receptors on the SMC plasma membrane and activate the phospholipase C (PLC)—inositol 1,4,5-trisphoshate (IP₃)—diacylglycerol (DAG) signal transduction pathway [norepinephrine (Haeusler and De Peyer 1989 Eur J Pharmacol 166, 175-182; Neild and Kotecha 1987 Circ Res 60, 791-795; Nelson et al. 1988 Nature 336, 382-385), histamine (Casteels and Suzuki 1980 Pfluegers Arch 387, 17-25, Gokina and Bevan 2000, Am J Physiol Heart Circ Physiol 278, H2094-H2104), 5-hydroxytyptamine (Neild and Kotecha 1987 Circ Res 60, 791-795), and UTP (Welsh and Brayden 2001, Am J Physiol Heart Circ Physiol 280, H2545-H2553)] are known to depolarize and constrict arterial SMCs (See FIG. 1). A current unresolved issue in vascular biology is the identification and characterization of the membrane channels responsible for agonist-induced depolarization of arterial smooth muscle.

Recently mammalian homologues of the Drosophila transient receptor potential (TRP) channel have been identified in native vascular smooth muscle cells (SMCs). TRPC3 and TRPC6 channels, 2 of the family of TRP channels, are activated by DAG independent of PKC (Venkatachalam et al 2003 J Biol Chem 278, 29031-29040) and give rise to a cation current that has relatively low selectivity for Ca²⁺ over Na⁺ (Hofmann et al 1999 Nature 397, 259-263). Recently, TRPC6 channels were reported to mediate a nonselective cation current activated by α₁-adrenergic receptor stimulation in rabbit portal vein SMC's (Inoue et al 2001, Circ Res 88, 325-332) and in rat embryonic aorta smooth muscle cells exposed to vasopressin (Jung et al 2002 Am J Physiol Cell Physiol 282, C347-C359). A clear role for TRPC6 regulation of myogenic tone in rat cerebrovascular resistance arteries has also been demonstrated (Welsh et al 2002 Circ Res 90, 248-250). However, in contrast to TRPC6, a role for TRPC3 channels in native vascular SMCs has not been established.

### SUMMARY OF THE INVENTION

The present invention is based at least in part on the discovery that TRPC3 channels are involved in ion fluxes that control arterial diameter and that modulation of these channels can be performed in order to therapeutically regulate arterial diameter. It was observed, as described in more detail below, that suppression of TRPC3, but not TRPC6, attenuated UTP-induced depolarization and constriction of cerebral arteries and abolished a UTP activated ion current in isolated arterial SMCs, demonstrating that TRPC3 is specifically involved in agonist-evoked arterial SMC constriction. It was also observed that UTP-induced vasoconstriction is maintained only in the presence of extracellular Ca²⁺ and that only a portion of the extracellular Ca²⁺ influx occurred through voltage-gated channels. Using antisense oligodeoxynucleotides to suppress TRPC3 expression; it was revealed that direct permeation of Ca²⁺ through TRPC3 channels is a significant Ca²⁺ influx pathway that contributes to UTP-induced constriction of pressurized cerebral arteries.

One aspect of the invention is a method for treating hypertension by administering to a subject an effective amount of a TRPC3 channel inhibitor for treating hypertension. In another aspect the invention is a method for reducing blood pressure in a subject by administering to a subject in need thereof an effective amount of a TRPC3 channel inhibitor for reducing blood pressure in the subject.

The TRPC3 channel inhibitor may be an activity inhibitor in some embodiments. The activity inhibitor may be a small molecule. In other embodiments the TRPC3 channel inhibitor may be an expression inhibitor. The expression inhibitor in some embodiments is an antisense or siRNA molecule.

In some embodiments the subject has or is at risk of developing a vasospasm. The vasospasm may in some embodiments be a cerebral vasospasm, a coronary artery vasospasm or a vasospasm associated with vascular surgery.

The TRPC3 channel inhibitor may be administered by any known route to the subject, for instance, orally, intravenously, or an intra-arterial route.

In other aspects the invention is a composition, including a TRPC3 channel inhibitor and an anti-hypertensive drug formulated with a pharmaceutically-acceptable carrier. In some embodiments the TRPC3 channel inhibitor is an activity inhibitor. In other embodiments the TRPC3 channel inhibitor is an expression inhibitor, such as an antisense or siRNA molecule. In yet other embodiments the composition is formulated for administration by an oral, intravenous, or intra-arterial route.

In other aspects of the invention a kit is provided. The kit includes a container housing an TRPC3 channel inhibitor and instructions for administering the TRPC3 channel inhibitor to a subject in order to lower the blood pressure of the subject. In some embodiments the subject has hypertension or vasospasm.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

### BRIEF DESCRIPTION OF DRAWINGS

This application includes examples which refer to figures or other drawings. It is to be understood that the referenced figures are illustrative only and are not essential to the enablement of the claimed invention.

FIG. 1 is a picture illustrating a model for agonist-induced membrane depolarization and constriction mediated by TRPC3 channels in arterial smooth muscle cells.

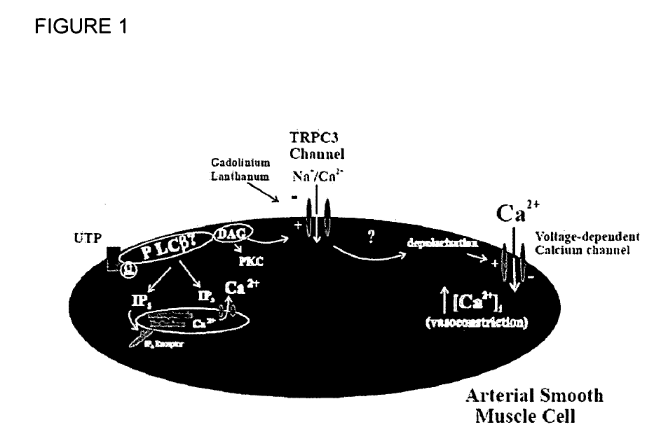

FIG. 2 shows that TRPC3 is expressed in rat cerebral artery vascular smooth muscle. FIG. 2A is a gel image showing that TRPC3 mRNA was detected in cerebral artery and vascular smooth muscle cells (VSM) using RT-PCR. (+) and (−) indicate PCR reactions run with and without reverse transcriptase in order to verify the absence of genomic DNA contamination. FIG. 2B is a gel image showing Western blots of cerebral artery samples exposed to TRPC3 antibody (Ab) in the presence or absence of TRPC3 antigenic peptide. The band with a molecular weight of approximately 120 kDa was not apparent in the presence of the antigenic peptide. FIG. 2C is an image showing immunofluorescent staining of cerebral artery. The left panel shows positive staining for the anti-TRPC3 antibody in gray. The right panel shows a lack of positive staining for the anti-TRPC3 antibody in the presence of the antigen peptide. The dotted white line defines the outer diameter of the arterial segment.

FIG. 3 demonstrates that reversible permeabilization (R-P) enhances oligodeoxynucleotide (ODN) uptake and ODNs suppress TRPC3 expression in rat cerebral artery. FIG. 3A is an image showing entry of fluorescein-labeled ODNs is greatly enhanced by reversible permeabilization (R-P) when compared to arteries exposed to ODNs in phosphate buffered saline (PBS) for an equivalent period of time. FIG. 3B is gel images from Western blots showing the effect of TRPC3 antisense oligodeoxynucleotides on TRPC3, TRPC6 and glyceraldehyde dehydrogenase (GAPDH) protein expression in cerebral artery. FIG. 3C is a graph showing summary data for the effect of TRPC3 sense and antisense ODNs on TRPC3 (left panel) and TRPC6 (right panel) protein expression in rat cerebral artery. The TRPC to GAPDH band density ratio was determined for each sense and antisense sample and then multiplied by 100 to yield a whole number. All values are the mean±SEM. The asterisk (*) indicates a significant difference (p≦0.05) in the TRPC to GAPDH ratio between sense (n=4) and antisense (n=4) samples.

FIG. 4 shows that suppression of TRPC3 expression with anti-sense oligodeoxynucleotides attenuates uridine triphosphate (UTP)—induced membrane depolarization and vasoconstriction. FIG. 4A is a graph of summary data showing that greater membrane depolarization occurs in sense- (n=6) than antisense- (n=5) treated cerebral arteries exposed to increasing concentrations of UTP. Resting membrane potentials (Vm) were −50±1 mV and −49±2 mV in sense- and antisense-treated arteries, respectively. FIG. 4B is a graph of summary data showing the decrease in vessel diameter (Percent Constriction) of TRPC3 sense- (n=4) and TRPC3 antisense- (n=4) treated arteries exposed to increasing concentrations of UTP. Initial internal diameters were 173±16 μm in sense-treated and 187±15 μm in antisense-treated arteries (no significant difference). All values are the mean±SEM. The asterisk (*) indicates a significant difference (p≦0.05) between sense- and antisense-treated vessels.

FIG. 5 shows that pressure-induced depolarization and myogenic tone were identical in TRPC3 sense- and antisense-treated arteries. The arterial SMCs depolarized by 14±1 mV in both TRPC3 sense (n=6) and TRPC3 antisense (n=7) arteries, and developed 26±4% (TRPC3 sense) and 27±5% (TRPC3 antisense) myogenic tone when intravascular pressure was increased from 20 to 80 mmHg.

FIG. 6 shows that TRPC3 antisense suppresses UTP-induced currents in freshly isolated VSM (Vascular Smooth Muscle) cells. Cells were patched in the perforated-patch configuration and whole-cell currents were recorded during voltage ramps from −120 to +20 mV from a holding potential of −60 mV. Examples of ramp currents in the absence (Control) and presence of UTP for cells from sense (FIG. 6A) and antisense (FIG. 6B) treated vessels. FIG. 6C is a graph showing mean±SEM of UTP-induced current density (difference currents) at −120 mV. The asterisk (*) indicates a significant difference (p≦0.05) between TRPC3 sense (n=9) and TRPC3 antisense-treated (n=5) SMCs.

FIG. 7 shows that extracellular Ca²⁺ entry through voltage-dependent Ca²⁺ channels contributes to uridine triphosphate (UTP)—induced vasoconstriction in pressurized rat cerebral arteries. FIG. 7A is a graph of original diameter recordings showing that 1 μM nisoldopine (NIS), an L-type Ca²⁺ antagonist, partially reverses the constriction induced by UTP in TRPC3 sense-, but not in TRPC3 antisense-treated arteries. Paired TRPC3 sense- and antisense-treated arteries were exposed to 1 μM UTP resulting in a 38% constriction in the sense-treated artery and a 30% constriction in the antisense-treated artery. FIG. 7B is a graph of a summary of the effects of NIS on UTP-induced tone in arteries exposed to TRPC3 sense (n=10) or TRPC3 antisense (n=8) oligodeoxynucleotides. The initial lumenal diameter was 189±15 μm for sense-treated and 170±15 μm for antisense-treated. Values are expressed as percent dilation that was calculated as follows:

$$\% dilation = \frac{ϕ ( UTP - NIS ) - ϕ ( UTP )}{ϕ ( initial ) - ϕ ( UTP )} \times 100 , where ϕ = arterial diameter$$

Values are the mean±SEM. The asterisk (*) indicates a significant difference (p≦0.05) between TRPC3 sense- and TRPC3 antisense-treated arteries.

FIG. 8 shows the relative amount of TRPC channel proteins in resistance arteries in various vascular beds from 15-18 week old spontaneously hypertensive rats (SHR) and Wistar-Kyoto control rats (WKY). FIG. 8A shows the expression of TRPC1, while FIG. 8B and 8C show the expression of TRPC3 and TRPC6 respectively (n=3−5).

FIG. 9 shows the relative amount of TRPC channel proteins in resistance arteries in various vascular beds from angiotensin-I1 hypersensitive rats treated with angiotensin-II or treated with a control. FIG. 9A shows the expression of TRPC1, while FIG. 9B and 9C show the expression of TRPC3 and TRPC6 respectively (n=3−5).

FIG. 10 shows the change in global intracellular [Ca²⁺] and artery constriction in response to OAG (1-oleyol-2-acetyl-sn-glycerol, an analogue of diacylglycerol) in intact functional cerebral arteries harvested from Sprague Dawley, WKY and SHR rats. FIG. 10A shows [Ca²⁺] increase, while FIG. 10B shows the increase of the artery constriction.

FIG. 11 shows that UTP-induced Ca²⁺ elevation and vasoconstriction are not maintained in the absence of extracellular Ca²⁺. FIG. 11A: Original tracings of arterial wall Ca²⁺ (top) and lumen diameter (bottom) recorded simultaneously from a pressurized (20 mm Hg) artery. The artery was initially superfused with a physiological saline solution (PSS) containing 1.6 mM Ca²⁺ (white background) and exposed to 30 μM UTP. Once the artery wall Ca²⁺ and lumen diameter reached a steady state response to UTP, extracellular Ca²⁺ was removed by superfusing the artery with Ca²⁺ free PSS (grey background). Extracellular Ca²⁺ was reintroduced to the artery in the presence of UTP by superfusing the artery with PSS (white background). FIG. 11B:Summary data for n=8 arteries. The measured arterial wall Ca²⁺ concentration ([Ca²⁺]ᵢ nM) is presented in the left panel and the measured arterial diameter (Diameter μm) is presented in the right panel. 1^{st} UTP in PSS represents the exposure of the artery to UTP prior to removal of extracellular Ca²⁺ from the superfusate and 2^{nd} UTP in PSS represents the exposure of the artery to UTP following the period of superfusion of the artery with Ca²⁺ free PSS. All values are the mean±SEM and represent the steady state average over the last 30 seconds of a condition. *: significantly different (p<0.05) from the Resting in PSS value. **: significantly different (p<0.05) from both the Resting in PSS and Resting in Ca²⁺ free values (n=8).

FIG. 12 shows that UTP-induces Ca²⁺ influx and vasoconstriction with L-type Ca²⁺ channels blocked. FIG. 12A: Arterial wall Ca²⁺ concentration (upper panel) and lumen diameter (lower panel) were simultaneously recorded from pressurized (20 mmHg) cerebral arteries. *: significantly different (p<0.05) from Ca²⁺ free values, with or without UTP. **: significantly different (p<0.05) form nimodipine plus Ca²⁺ plus UTP value (n=8).

FIG. 13 shows that Gd⁺³ inhibits extracellular Ca²⁺ influx and UTP-induced vasoconstriction of rat cerebral arteries. FIG. 13A: Original tracings of arterial wall Ca²⁺ (top) and lumen diameter (bottom) simultaneously recorded from a pressurized (20 mm Hg) artery. Nimodipine was present throughout to block Ca²⁺ entry through L-type Ca²⁺ channels. UTP and Gd³⁺ were present in the superfusate during the times indicated. FIG. 13B: Summary Data of arterial wall Ca²⁺ concentration (left panel) and arterial diameter (right panel) for arteries treated with UTP and Gd³⁺. *: significantly different (p<0.05) from Ca²⁺ free values. **: significantly different (p<0.05) from Ca²⁺ free value and Ca²⁺ plus UTP value (n=4).

FIG. 14 shows that UTP activates extracellular Ca²⁺ influx through TRPC3 channels. FIG. 14A: Original tracings of arterial wall Ca²⁺ (top) and lumen diameter (bottom) recorded from posterior cerebral arteries obtained from the same animal and treated with either TRPC3 sense (light grey) or antisense (dark grey) oligodeoxynucleotides (ODNs). Arteries were superfused with Ca²⁺ free PSS (grey background) or PSS containing 1.6 mM Ca²⁺ (white background). Nimodipine, UTP, and caffeine were present in the superfusate during the times indicated. FIG. 14B: Summary Data of arterial wall Ca²⁺ concentration (left panel) and arterial diameter (right panel) in the presence of UTP for arteries treated with TRPC3 sense or antisense ODNs. *: significantly different (p<0.05) from sense-treated arteries (n=7).

FIG. 15 shows that the diacylglycerol analog 1-oleoyl-2-acetyl-sn-glycerol (OAG), which is known to directly activate TRPC channels, increases arterial wall [Ca²⁺] and constricts TRPC3 sense- but not TRPC3 antisense-treated cerebral arteries. FIG. 15A: Original tracings of arterial wall Ca²⁺ (top) and lumen diameter (bottom) recorded from pressurized (20 mm Hg) cerebral arteries treated with either TRPC3 sense (light grey) or antisense (dark grey) oligodeoxynucleotides (ODNs). Arteries were superfused with PSS containing 1.6 mM Ca²⁺ (white background) and treated with 300 μM OAG as indicated. FIG. 15B: Summary Data of arterial wall Ca²⁺ concentration (left panel) and arterial diameter (right panel) in the presence of OAG for arteries treated with TRPC3 sense or antisense ODNs. *: significantly different (p<0.05) from sense-treated arteries (n=18 for sense-treated arteries; n=6 for antisense-treated arteries).

### DETAILED DESCRIPTION

Transient receptor potential (TRP) cation channels are present in vascular smooth muscle and are involved in the smooth muscle depolarizing response to stimuli such as membrane stretch. It was discovered according to aspects of the invention that a member of the TRPC subfamily of TRP channels, i.e. the TRPC3 channel, mediates depolarization of vascular smooth muscle induced by receptor activation.

UTP invokes membrane depolarization and constriction of vascular smooth muscle by activating a cation current that exhibits inward rectification, is not rapidly desensitized, and is blocked by Gd³⁺. The molecular identity of this UTP-induced cation current has not been determined. Canonical transient receptor potential (TRPC) proteins form Ca²⁺ permeable, non-selective cation channels in a variety of mammalian tissues. Suppression of one member of this family of channels, TRPC6, has been reported to prevent an α1—adenoreceptor-activated cation current in cultured rabbit portal vein myocytes. However, suppression of TRPC6 channels in cerebral vascular smooth muscle, did not attenuate the UTP-induced membrane depolarization and vasoconstriction. In contrast, TRPC3, unlike TRPC6, was found to mediate the agonist induced depolarization, as observed in rat cerebral artery following UTP activation of the P2Y receptor.

Thus, TRPC3 channels in vascular smooth muscle mediate agonist-induced depolarization which contributes to vasoconstriction in resistance-sized cerebral arteries. The data described in more detail below supports these findings. For instance, the role of TRPC3 channels in these responses was demonstrated using antisense oligodeoxynucleotides to suppress channel expression. Western blots of arterial lysates indicated that TRPC3 expression was reduced by nearly 60% in the TRPC3 antisense compared with sense treated arteries. UTP-induced depolarizations were significantly reduced in TRPC3-anitsense versus TRPC3-sense treated arteries, whereas depolarizations induced by elevation of intravascular pressure were identical between the two groups. Constrictions in response to low concentration of UTP (0.1 to 1.0 μM) were significantly less in antisense versus sense treated arteries. These findings indicate that TRPC3 channels are important contributors to the cerebrovascular smooth muscle depolarization and contraction induced by UTP.

The invention is based at least in part on the discovery that TRPC3 is expressed in native rat cerebral artery and that TRPC3 suppression inhibits UTP-induced cation currents and Ca²⁺ influx in vascular smooth muscle cells but does not affect pressure-induced depolarization or constriction in rat cerebral artery. TRPC3 channel inhibitors that reduce TRPC3 channel activity reduce blood pressure in vivo and are useful as anti-hypertensive agents as well as therapeutics in the treatment of vasospasm.

The methods of the invention are useful for treating a subject in need thereof. A subject in need thereof is a subject having or at risk of having high blood pressure, hypertension or vasospasm. In its broadest sense, the terms “treatment” or “to treat” refer to both therapeutic and prophylactic treatments. If the subject in need of treatment is experiencing a condition (i.e., has or is having a particular condition), then “treating the condition” refers to ameliorating, reducing or eliminating one or more symptoms arising from the condition. In some embodiments, treating the condition refers to ameliorating, reducing or eliminating a specific symptom or a specific subset of symptoms associated with the disorder. If the subject in need of treatment is one who is at risk of having a condition, then treating the subject refers to reducing the risk of the subject having the condition.

A “subject having hypertension” is a subject who has a disorder involving elevated arterial pressure. A “subject at risk of developing hypertension” is a subject who has a propensity of developing hypertension because of certain factors affecting the cardiovascular system of the subject. Factors which influence the development of hypertension include but are not limited to exposure to environmental factors such as high salt intake, occupation, and alcohol; as well as obesity and heredity. It is desirable to reduce the risk in these subjects of developing hypertension. Reducing the risk of hypertension includes a slowing of the progression towards hypertension or preventing the development of hypertension. In some embodiments the subject having hypertension is one that has cardiac hypertrophy and/or heart disease. In other embodiments the subject having hypertension is one that does not have cardiac hypertrophy and/or heart disease.

While yet the subject of extensive research, hypertension appears to be the product of an inherited predisposition—coupled with dietary, emotional, and environmental factors, which results in a structural adaptation of the cardiac muscle and the large blood vessels. Most patients display heightened vascular and cardiac reactions to sympathetic nervous stimulation, but the precise relationship of sympathetic nervous stimulation to the etiology of the disease. Nevertheless, hypertension results in chronic readjustment of cardiovascular hemodynamics, alteration of blood vessel walls, cardiovascular resistance and regional transmural pressures.

Pharmacologic management of hypertension is generally directed to the normalization of altered hemodynamic parameters, and many drugs and drug classes, either as monotherapy or in combination treatment, can reduce and control elevated blood pressure. However, treatment of hypertension does not always correspondingly benefit the morbidity and mortality of the condition, either because chronic hypertension has produced other significant and irreversible cardiovascular changes, or because present drugs have an adverse effect on some other risk factor for cardiovascular disease. Rather, current drug therapy simply provides sustained arterial pressure reduction.

Pulmonary hypertension is a disease characterized by increased pulmonary arterial pressure and pulmonary vascular resistance of the vessels, as well as vascular remodeling which leads to narrowed lumens of the vessels. Pulmonary hypertension can be primary, i.e. of unknown or unidentifiable cause, or can be secondary to a known cause such as hypoxia or congenital heart shunts. The term “primary pulmonary hypertension” generally refers to a condition in which there is elevated arterial pressures in the small pulmonary arteries. Pulmonary hypertension generally occurs independently of and is unrelated to systemic hypertension. In vitro studies have concluded that changes in Ca⁺⁺ concentrations may be involved in pulmonary tissue damage associated with pulmonary hypertension. (Farruck et al 1992 Am Rev Respir Dis 145, 1389-1397). A subject having pulmonary hypertension as used herein is a subject having a right ventricular systolic or a pulmonary artery systolic pressure, at rest, of at least 20 mmHg. Pulmonary hypertension is measured using conventional procedures well-known to those of ordinary skill in the art.

A subject at risk of developing pulmonary hypertension may be treated prophylactically to reduce the risk of pulmonary hypertension. A subject with an abnormally elevated risk of pulmonary hypertension is a subject with chronic exposure to hypoxic conditions, a subject with sustained vasoconstriction, a subject with multiple pulmonary emboli, a subject with cardiomegaly and/or a subject with a family history of pulmonary hypertension.

A vasospasm is a sudden decrease in the internal diameter of a blood vessel that results from contraction of smooth muscle within the wall of the vessel. Vasospasms result in decreased blood flow, but increased system vascular resistance.

A subject having a coronary artery vasospasm is one who has symptoms of or has been diagnosed with coronary artery vasospasm. A subject at risk of coronary artery vasospasm is one who has one or more predisposing factors to the development of cerebral vasospams. Examples of predisposing factors are cigarette use or vasospastic disorders such as Raynaud's phenomenon and migraine headaches.

Coronary arterial spasm can occur in the absence of significant coronary atherosclerosis and is thought to be an initiating event in variant angina and in myocardial infarction. Coronary spasm may occur without the patient feeling any significant discomfort. In an electrically unstable heart, diverse neural impulses to the heart may provoke coronary vascular spasm. This may result in enhanced myocardial ischemia and arrhythmia, which in turn may culminate in ventricular fibrillation and sudden cardiac death. As in variant or vasospastic angina, the calcium channel antagonists may be of particular usefulness due to their effect on cardiac and vascular smooth muscle. “Peripheral vascular disorder” is a disorder caused by segmental lesions arising from stenosis or occlusion of large and medium size blood vessels, and most often occurs in the upper extremities.

A subject having a cerebral vasospasm is one who has symptoms of or has been diagnosed with cerebral vasospasm. A subject at risk of cerebral vasospasm is one who has one or more predisposing factors to the development of cerebral vasospams. An example of a predisposing factor is existence of a subarachnoid hemorrhage. A subject who has experienced a recent subarachnoid hemorrhage is at significantly higher risk of developing cerebral vasospasm than a subject who has not had a recent subarachnoid hemorrhage.

“Subarachnoid hemorrhage” (SAH) is a condition in which blood collects beneath the arachnoid mater, a membrane that covers the brain. This area, called the subarachnoid space, normally contains cerebrospinal fluid. The accumulation of blood in the subarachnoid space can lead to stroke, seizures, and other complications. Additionally, subarachnoid hemorrhages may cause permanent brain damage and a number of harmful biochemical events in the brain. The term “subarachnoid hemorrhage” is used herein to refer to non-traumatic types of hemorrhages non-traumatic types of hemorrhages, usually caused by rupture of a berry aneurysm or arteriovenous malformation (AVM). Other causes include bleeding from a vascular anomaly and extension into the subarachnoid space from a primary intracerebral hemorrhage. Symptoms of subarachnoid hemorrhage include sudden and severe headache, nausea and/or vomiting, symptoms of meningeal irritation (e.g., neck stiffness, low back pain, bilateral leg pain) photophobia and visual changes, and/or loss of consciousness.

Subarachnoid hemorrhage is often secondary to a head injury or a blood vessel defect known as an aneurysm. In some instances, subarachnoid hemorrhage can induce a cerebral vasospasm that may in turn lead to an ischemic stroke. A common manifestation of a subarachnoid hemorrhage is the presence of blood in the CSF.

Subjects having a subarachnoid hemorrhage can be identified by a number of symptoms. For example, a subject having a subarachnoid hemorrhage will present with blood in the subarachnoid, usually in a large amount. Subjects having a subarachnoid hemorrhage can also be identified by an intracranial pressure that approximates mean arterial pressure, by a fall in cerebral perfusion pressure or by the sudden transient loss of consciousness (sometimes preceded by a painful headache). In about half of cases, subjects present with a severe headache which may be associated with physical exertion. Other symptoms associated with subarachnoid hemorrhage include nausea, vomiting, memory loss, hemiparesis and aphasia. Subjects having a subarachnoid hemorrhage can also be identified by the presence of creatine kinase-BB isoenzyme activity in their CSF. This enzyme is enriched in the brain but is normally not present in the CSF. Thus, its presence in the CSF is indicative of “leak” from the brain into the subarachnoid. Assay of creatine-kinase BB isoenzyme activity in the CSF is described by Coplin et al. (Coplin et al 1999 Arch Neurol 56, 1348-1352) Additionally, a spinal tap or lumbar puncture can be used to demonstrate if there is blood present in the CSF, a strong indication of a subarachnoid hemorrhage. A cranial CT scan or an MRI can also be used to identify blood in the subarachnoid region. Angiography can also be used to determine not only whether a hemorrhage has occurred but also the location of the hemorrhage.

Subarachnoid hemorrhage commonly results from rupture of an intracranial saccular aneurysm or from malformation of the arteriovenous system in, and leading to, the brain. Accordingly, a subject at risk of having a subarachnoid hemorrhage includes subjects having a saccular aneurysm as well as subjects having a malformation of the arteriovenous system. It is estimated that 5% of the population have such aneurysms yet only 1 in 10,000 people actually have a subarachnoid hemorrhage. The top of the basilar artery and the junction of the basilar artery with the superior cerebellar or the anterior inferior cerebellar artery are common sites of saccular aneurysms. Subjects having a subarachnoid hemorrhage may be identified by an eye examination, whereby slowed eye movement may indicate brain damage. A subject with a developing saccular aneurysm can be identified through routine medical imaging techniques, such as CT and MRI. A developing aneurysm forms a mushroom-like shape (sometimes referred to as “a dome with a neck” shape).

A vasospasm is a sudden decrease in the internal diameter of a blood vessel that results from contraction of smooth muscle within the wall of the vessel. Vasospasms result in decreased blood flow, but increased system vascular resistance. It is generally believed that vasospasm is caused by local injury to vessels, such as that which results from atherosclerosis and other structural injury including traumatic head injury. Cerebral vasospasm is a naturally occurring vasoconstriction which can also be triggered by the presence of blood in the CSF, a common occurrence after rupture of an aneurysm or following traumatic head injury. Cerebral vasospasm can ultimately lead to brain cell damage, in the form of cerebral ischemia and infarction, due to interrupted blood supply.

Cerebral vasospasm is characterized by a sudden decrease in the internal diameter of a blood vessel that results from contraction of smooth muscle within the wall of the vessel. This causes a decrease in blood flow, but an increase in systemic vascular resistance. As used herein, cerebral vasospasm refers to the delayed occurrence of narrowing of large capacity arteries at the base of the brain after subarachnoid hemorrhage, often associated with diminished perfusion in the territory distal to the affected vessel. Cerebral vasospasm can occur any time after rupture of an aneurysm but most commonly peaks at seven days following the hemorrhage and often resolves within 14 days when the blood has been absorbed by the body.

A subject having a vasospasm is a subject who presents with diagnostic markers and symptoms associated with vasospasm. Diagnostic markers include the presence of blood in the CSF and/or a recent history of a subarachnoid hemorrhage. Vasospasm associated symptoms include paralysis on one side of the body, inability to vocalize the words or to understand spoken or written words, and inability to perform tasks requiring spatial analysis. Such symptoms may develop over a few days, or they may fluctuate in their appearance, or they may present abruptly.

MR angiography and CT angiography can be used to diagnose cerebral vasospasm. Angiography is a technique in which a contrast agent is introduced into the blood stream in order to view blood flow and/or arteries. A contrast agent is required because blood flow and/or arteries are sometimes only weakly apparent in a regular MR or CT scan. Appropriate contrast agents will vary depending upon the imaging technique used. For example, gadolinium is a common contrast agent used in MR scans. Other MR appropriate contrast agents are known in the art. Transcranial Doppler ultrasound can also be used to diagnose and monitor the progression of a vasospasm. As mentioned earlier, the presence of blood in the cerebrospinal fluid can be detected using CT scans. However, in some instances where the amount of blood is so small as to not be detected by CT, a lumbar puncture is warranted.

A subject at risk of a vasospasm includes a subject who has detectable blood in the cerebrospinal fluid, or one who has a detectable aneurysm as detected by a CT scan, yet has not begun to experience the symptoms associated with having a vasospasm. A subject at risk of a vasospasm may also one who has experienced a traumatic head injury. Traumatic head injury usually results from a physical force to the head region, in the form of a fall or a forceful contact with a solid object. Subjects at risk of a vasospasm may also include those who have recently (e.g., in the last two weeks or months) experienced a subarachnoid hemorrhage (as described above).

As used herein, a subject includes humans, non human primates, dogs, cats, sheep, goats, cows, pigs, horses and rodents. In preferred embodiments, the subject is human. In some embodiments the subject is free of disorders otherwise calling for treatment with TRPC3 channel inhibitors.

The invention provides methods and compositions to treat conditions which would benefit from, and which thus can be treated by, an inhibition of ion flux across TRPC3 channels. Such compounds are referred to as TRPC3 channel inhibitors.

Calcium signaling has been implicated in the regulation of a variety of cellular responses, such as neuronal development and maintenance, and cell growth and differentiation. There are two general methods by which intracellular concentrations of calcium ions may be increased: calcium ions may be brought into the cell from the extracellular milieu through the use of specific channels in the cellular membrane, or calcium ions may be freed from intracellular stores, again being transported by specific membrane channels in the storage organelle.

The TRP channel family is a member of the calcium channel group. These channels include transient receptor potential protein and homologues thereof, the vanilloid receptor subtype I, stretch-inhibitable non-selective cation channel, olfactory, mechanosensitive channel, insulin-like growth factor I-regulated calcium channel, and vitamin D-responsive apical, epithelial calcium channel (ECaC). Each of these molecules is at least 700 amino acids, and shares certain conserved structural features. Predominant among these structural features are six transmembrane domains, with an additional hydrophobic loop present between the fifth and sixth transmembrane domains. It is believed that this loop is integral to the activity of the pore of the channel formed upon membrane insertion. TRP channel proteins also include one or more ankyrin domains and frequently display a proline-rich region at the N-terminus.

The TRP1 channel family comprises a large group of channels mediating an array of signal and sensory transduction pathways. The proteins of the mammalian TRPC subfamily are the products of at least seven genes coding for cation channels that appear to be activated in response to PLC-coupled receptors. The putative ion channel subunits TRPC3, TRPC6, and TRPC7 comprise a structurally related subgroup of the family of mammalian TRPC channels. The ion channels formed by these proteins appear to be activated downstream of phospholipase C (PLC). PLC-dependent activation of TRPC6 and TRPC7 has been shown to involve diacylglycerol and is independent of G proteins or inositol 1,4,5-triphosphate (IP3).

TRPC channels are widely expressed among cell types and may play important roles in receptor-mediated Ca²⁺ signaling. The TRPC3 channel is known to be a Ca²⁺-conducting channel activated in response to phospholipase C-coupled receptors. TRPC3 channels have been shown to interact directly with intracellular inositol 1,4,5-trisphosphate receptors (InsP₃Rs) and that channel activation is mediated through coupling to InsP₃Rs.

Agents useful for increasing arterial blood flow, inhibiting vasoconstriction or inducing vasodilation are agents which inhibit TRPC3 channels. These inhibitors embrace compounds which are TRPC3 channel antagonists. Such inhibitors are referred to as activity inhibitors or TRPC3 channel activity inhibitors. As used herein, an activity inhibitor is an agent which interferes with or prevents the activity of an TRPC3 channel. An activity inhibitor may interfere with the ability of the TRPC3 channel to bind an agonist such as UTP. An activity inhibitor may be an agent which competes with a naturally occurring activator of TRPC3 channel for interaction with the activation binding site on the TRPC3 channel. Alternatively, the activity inhibitor may bind to the TRPC3 channel at a site distinct from the activation binding site, but in doing so, it may, for example, cause a conformational change in the TRPC3 channel which is transduced to the activation binding site, thereby precluding binding of the natural activator. Alternatively, an activity inhibitor may interfere with a component upstream or downstream of the TRPC3 channel but which interferes with the activity of the TRPC3 channel. This latter type of activity inhibitor is referred to as a functional antagonist. Non-limiting examples of a TRPC3 channel inhibitor which is an activity inhibitor are gadolinium chloride and lanthanum chloride.

Other agents which are useful according to the methods of the invention in the treatment of conditions described herein include agents which interfere with TRPC3 channel expression at either the mRNA or protein level. Such inhibitors are referred to as expression inhibitors or TRPC3 channel expression inhibitors. Expression inhibitors are described in more detail below.

The inhibitors described herein are isolated molecules. An isolated molecule is a molecule that is substantially pure and is free of other substances with which it is ordinarily found in nature or in vivo systems to an extent practical and appropriate for its intended use. In particular, the molecular species are sufficiently pure and are sufficiently free from other biological constituents of host cells so as to be useful in, for example, producing pharmaceutical preparations or sequencing if the molecular species is a nucleic acid, peptide, or polysaccharide. Because an isolated molecular species of the invention may be admixed with a pharmaceutically-acceptable carrier in a pharmaceutical preparation, the molecular species may comprise only a small percentage by weight of the preparation. The molecular species is nonetheless substantially pure in that it has been substantially separated from the substances with which it may be associated in living systems.

In one aspect of the invention, a TRPC3 channel inhibitor is administered to the subject having or at risk of having hypertension or a vasospasm in an effective amount to treat hypertension or the vasospasm. An effective amount to treat hypertension or a vasospasm may be that amount necessary to ameliorate, reduce or eliminate altogether one or more symptoms relating to hypertension or a vasospasm, preferably including brain damage that results from vasospasm such as an infarct. Brain damage can be measured anatomically using medical imaging techniques to measure infarct sizes. Alternatively or in conjunction, brain damage may be measured functionally in terms of cognitive or sensory skills of the subject.

Inhibitors can be combined with other therapeutic agents, such as an anti-hypertensive agent and an anti-cerebral vasospasm drug. The inhibitor and other therapeutic agent may be administered simultaneously or sequentially. When the other therapeutic agents are administered simultaneously they can be administered in the same or separate formulations, but are administered at the same time. The other therapeutic agents are administered sequentially with one another and with inhibitor, when the administration of the other therapeutic agents and the inhibitor is temporally separated. The separation in time between the administration of these compounds may be a matter of minutes or it may be longer.

In one embodiment the method includes the step of administering a medicament other than the compound for the treatment of cardiovascular disease. Preferably the medicament is for treating hypertension, such as an anti-hypertensive agent or drug An anti-hypertensive agent or drug may include, for example any one or more of Ajmaline; g-Aminobutyric acid; Alfuzosin Hydrochloride; Alipamide; Althiazide; Amiquinsin Hydrochloride; Amlodipine Besylate; Amlodipine Maleate; Amosulalol; Anaritide Acetate; Aryloxypropanolamine derivatives; Atiprosin Maleate; Belfosdil; Bemitradine; Bendacalol Mesylate; Bendroflumethiazide; Benzothiadiazine derivatives; Benzthiazide; Betaxolol Hydrochloride; Bethanidine Sulfate; Bevantolol Hydrochloride; Biclodil Hydrochloride; Bisoprolol; Bisoprolol Fumarate; Bucindolol Hydrochloride; Bupicomide; Bufeniode; Bufuralol; Buthiazide: Candoxatril; Candoxatrilat; Captopril; N-Carboxyalkyl derivatives; Carvedilol; Ceronapril; Chlorothiazide Sodium; Chlorthalidone; Cicletanine; Ciclasidomine; Cilazapril; Clonidine; Clonidine Hydrochloride; Clopamide; Cyclopenthiazide; Cyclothiazide; Cyptenamine tannates; Darodipine; Debrisoquin Sulfate; Delapril Hydrochloride; Diapamide; Diazoxide; Dilevalol Hydrochloride; Diltiazem Malate; Ditekiren; Doxazosin Mesylate; Ecadotril; Enalapril Maleate; Enalaprilat; Enalkiren; Endralazine Mesylate; Epithiazide; Eprosartan; Eprosartan Mesylate; Fenoldopam Mesylate; Flavodilol Maleate; Flordipine; Flosequinan; Fosinopril Sodium; Fosinoprilat; Guanabenz; Guanabenz Acetate; Guanacline Sulfate; Guanadrel Sulfate; Guanazodine; Guancydine; Guanethidine Monosulfate; Guanethidine Sulfate; Guanfacine Hydrochloride; Guanisoquin Sulfate; Guanoclor Sulfate; Guanoctine Hydrochloride; Guanoxabenz; Guanoxan Sulfate; Guanoxyfen Sulfate; Hydralazine Hydrochloride; Hydrazines and phthalazines; Hydralazine Polistirex; Hydroflumethiazide; Imidazole derivatives; Indacrinone; Indapamide; Indolapril Hydrochloride; Indoramin; Indoramin Hydrochloride; Indorenate Hydrochloride; Ketanserin; Labetalol; Lacidipine; Leniquinsin; Levcromakalim; Lisinopril; Lofexidine Hydrochloride; Losartan Potassium; Losulazine Hydrochloride; Mebutamate; Mecamylamine Hydrochloride; Medroxalol; Medroxalol Hydrochloride; Methalthiazide; Methyclothiazide; Methyldopa; Methyldopate Hydrochloride; Methyl 4 pyridyl ketone thiosemicarbarzone; Metipranolol; Metolazone; Metoprolol Fumarate; Metoprolol Succinate; Metyrosine; Minoxidil; Monatepil Maleate; Muzolimine; Nebivolol; Nitrendipine; Ofornine; Pargyline Hydrochloride; Pazoxide; Pelanserin Hydrochloride; Perindopril Erbumine; Pempidine; Piperoxan; primaperone; Protoveratrines; Raubasine; Rescimetol; Rilemenidene; Pronethalol; Phenoxybenzamine Hydrochloride; Pinacidil; Pivopril; Polythiazide; Prazosin Hydrochloride; Primidolol; Prizidilol Hydrochloride; Quaternary Ammonium Compounds; Quinazoline derivatives; Quinapril Hydrochloride; Quinaprilat; Quinazosin Hydrochloride; Quinelorane Hydrochloride; Quinpirole Hydrochloride; Quinuclium Bromide; Ramipril; Rauwolfia Serpentina; Reserpine; Saprisartan Potassium; Saralasin Acetate; Sodium Nitroprusside; Sotalol; Sulfinalol Hydrochloride; Sulfonamide derivatives; Tasosartan; Teludipine Hydrochloride; Temocapril Hydrochloride; Terazosin Hydrochloride; Terlakiren; Tiamenidine; Tiamenidine Hydrochloride; Ticrynafen; Tinabinol; Tiodazosin; Tipentosin Hydrochloride; Trichlormethiazide; Trimazosin Hydrochloride; Trimethaphan Camsylate; Trimoxamine Hydrochloride; Tripamide; Tyrosinase; Urapidil; Xipamide; Zankiren Hydrochloride; and Zofenoprilat Arginine.

Subjects at risk of vasospasm are currently administered a variety of preventative medications including L-type voltage-dependent calcium channel (L-type VDCC) inhibitors (e.g., nimodipine), phenylephrine, dopamine, as well as a combination of mannitol and hyperventilation. Some forms of prophylactic treatments aim to increase the cerebral perfusion pressure. In accordance with the present invention, any of these prophylactic therapies may be co-administered to a subject at risk of having a vasospasm along with the agents of the invention. Thus, other therapeutic agents include but are not limited to anti-cerebral vasospasm drug such as L-type VDCC and a phenylalkalamine such as verapamil, etc.

Nitrates affect direct endothelium-independent vasodilatation of the large coronary arteries. In addition, a reduction of preload occurs due to dilatation of venous capacitance vessels, resulting in a decrease in myocardial oxygen consumption. Nitrates act as an exogenous source of nitric oxide, which causes vascular smooth muscle relaxation and may have a modest effect on platelet aggregation and thrombosis.

Nitroglycerin (Nitrolingual, Nitrostat, Minitran, Nitro-Bid, Nitro-Dur) causes relaxation of vascular smooth muscle by stimulating intracellular cyclic GMP. The result is a decrease in blood pressure. Dosage forms include SL, TD, and IV preparations. The distinction between short-acting preparations for treatment of acute attacks and long-acting preparations for prevention of recurrent episodes is important.

Isosorbide dinitrate (Isordil, Sorbitrate) relaxes vascular smooth muscle by stimulating intracellular cyclic GMP. Decreases preload and afterload, causing decreased myocardial oxygen demand.

Nifedipine (Adalat, Adalat CC, Procardia, Procardia XL) is a prototypical dihydropyridine indicated for treatment of acute attacks and prevention of recurrent attacks.

Verapamil (Calan, Calan SR, Covera HS, Isoptin, Verelan) during depolarization, inhibits calcium ion from entering slow channels or voltage-sensitive areas of the vascular smooth muscle and myocardium.

Diltiazem (Cardizem, Cardizem CD, Dilacor, Dilacor XR, Tiazac) -during depolarization inhibits calcium ions from entering the slow channels and voltage-sensitive areas of vascular smooth muscle and myocardium.

The compositions are delivered in effective amounts. The term effective amount refers to the amount necessary or sufficient to realize a desired biologic effect. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is effective to treat the particular subject. The effective amount for any particular application can vary depending on such factors as the disease or condition being treated, the particular inhibitor being administered, the size of the subject, or the severity of the disease or condition. One of ordinary skill in the art can empirically determine the effective amount of a particular inhibitor and/or other therapeutic agent without necessitating undue experimentation. It is preferred generally that a maximum dose be used, that is, the highest safe dose according to some medical judgment. Multiple doses per day may be contemplated to achieve appropriate systemic levels of compounds. Appropriate systemic levels can be determined by, for example, measurement of the patient's peak or sustained plasma level of the drug. “Dose” and “dosage” are used interchangeably herein.

Generally, daily oral doses of active compounds will be from about 0.01 milligrams/kg per day to 1000 milligrams/kg per day. It is expected that oral doses in the range of 0.5 to 50 milligrams/kg, in one or several administrations per day, will yield the desired results. Dosage may be adjusted appropriately to achieve desired drug levels, local or systemic, depending upon the mode of administration. For example, it is expected that intravenous administration would be from an order to several orders of magnitude lower dose per day. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of compounds.

For any compound described herein the therapeutically effective amount can be initially determined from preliminary in vitro studies and/or animal models. A therapeutically effective dose can also be determined from human data for inhibitors which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

For use in therapy, an effective amount of the inhibitor can be administered to a subject by any mode that delivers the inhibitor to the desired surface. Administering the pharmaceutical composition of the present invention may be accomplished by any means known to the skilled artisan. Preferred routes of administration include but are not limited to oral, intrathecal, intra-arterial, direct bronchial application, parenteral (e.g. intravenous), intramuscular, intranasal, sublingual, intratracheal, inhalation, ocular, vaginal, and rectal, e.g., using a suppository. The inhibitors and other therapeutics may also be delivered to a subject during surgery to treat an underlying condition or side effect such as subarachnoid hemorrhage or peripheral vasospasm or during intra-arterial procedures.

For oral administration, the compounds (i.e., inhibitors, and other therapeutic agents) can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, i.e. EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline (Abuchowski and Davis 1981, “Soluble Polymer-Enzyme Adducts” In: Enzymes as Drugs, Hocenberg and Roberts, eds., Wiley-Interscience, New York, N.Y., pp. 367-383; Newmark et al 1982 J Appl Biochem 4,185-189). Other polymers that could be used are poly-1,3-dioxolane and poly-1,3,6-tioxocane. Preferred for pharmaceutical usage, as indicated above, are polyethylene glycol moieties.

For the component (or derivative) the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the inhibitor (or derivative) or by release of the biologically active material beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic i.e. powder; for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the inhibitor may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, a-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential non-ionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the inhibitor or derivative either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

Also contemplated herein is pulmonary delivery of the inhibitors. The inhibitor is delivered to the lungs of a mammal while inhaling and traverses across the lung epithelial lining to the blood stream. Other reports of inhaled molecules include: Adjei et al 1990 Pharm Research 7, 565-569; Adjei et al 1990 Intl J Pharm 63,135-144 (leuprolide acetate); Braquet et al 1989 J Cardio Pharm 13(suppl. 5),143-146 (endothelin-1); Hubbard et al 1989 Annals of Int Medicine III, 206-212 (a1-antitrypsin); Smith et al. 1989 J Clin Invest 84, 1145-1146 (a-1-proteinase); Oswein et al 1990 “Aerosolization of Proteins”, Proceedings of Symposium on Respiratory- Drug Delivery II, Keystone, Colo., March, (recombinant human growth hormone); Debs et al 1988 J Immunol 140, 3482-3488 (interferon-γ and tumor necrosis factor alpha) and Platz et al U.S. Pat. No. 5,284,656 (granulocyte colony stimulating factor). A method and composition for pulmonary delivery of drugs for systemic effect is described in U.S. Pat. No. 5,451,569, issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of inhibitor. Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. Chemically modified inhibitor may also be prepared in different formulations depending on the type of chemical modification or the type of device employed.

Formulations suitable for use with a nebulizer, either jet or ultrasonic, will typically comprise inhibitor dissolved in water at a concentration of about 0.1 to 25 mg of biologically active inhibitor per mL of solution. The formulation may also include a buffer and a simple sugar (e.g., for inhibitor stabilization and regulation of osmotic pressure). The nebulizer formulation may also contain a surfactant, to reduce or prevent surface induced aggregation of the inhibitor caused by atomization of the solution in forming the aerosol.

Formulations for use with a metered-dose inhaler device will generally comprise a finely divided powder containing the inhibitor suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing inhibitor (or derivative) and may also include a bulking agent, such as lactose, sorbitol, sucrose, or mannitol in amounts which facilitate dispersal of the powder from the device, e.g., 50 to 90% by weight of the formulation. The inhibitor (or derivative) should most advantageously be prepared in particulate form with an average particle size of less than 10 mm (or microns), most preferably 0.5 to 5 mm, for most effective delivery to the distal lung.

Nasal delivery of a pharmaceutical composition of the present invention is also contemplated. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions.

Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides. Suppositories are a solid dosage form of medication that can be delivered internally to a patient, human or animal by insertion of the solid dosage form directly to the an area of the body. Known types of suppositories include rectal, vaginal and urethral suppositories. Commonly used bases, which are commercially available for suppositories include PCCA Base MBK™ (Fatty Acid Base, PCCA), PCCA Base A™ (Polyglycol 1450 MW, NF, PCCA), PCCA Base F™ (Synthetic Cocoa Butter, PCCA), Wecobee® M, R, S, W (Vegetable Oil, Hydrogenated, tepan Company, Northfield, Ill.), Witepsol® H12, H15, W35 (Vegetable Oil, Hydrogenated), Hydrokote® M (Vegetable Oil, Hydrogenated, Abitec Corporation, Columbus, Ohio), COA Base (Fatty Acid Base, Spectrum Pharmacy Products, Tucson), Supposibase (PEG/Vegetable, Spectrum Pharmacy Products, Tucson), Base A, B, D, Polyethylene Glycols, Spectrum Pharmacy Products, Tucson), and Polybase (Polyethylene Glycol Blend, Gallipot, Inc., St.Paul, Minn.)

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer 1990 Science 249, 1527-1533, which is incorporated herein by reference.

The inhibitors and optionally other therapeutics may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The pharmaceutical compositions of the invention contain an effective amount of an inhibitor and optionally therapeutic agents included in a pharmaceutically-acceptable carrier. The term pharmaceutically-acceptable carrier means one or more compatible solid or liquid filler, diluents or encapsulating substances which are suitable for administration to a human or other vertebrate animal. The term carrier denotes an organic or inorganic ingredient, natural or synthetic, with which the active ingredient is combined to facilitate the application. The components of the pharmaceutical compositions also are capable of being commingled with the compounds of the present. invention, and with each other, in a manner such that there is no interaction which would substantially impair the desired pharmaceutical efficiency.

The therapeutic agent(s), including specifically but not limited to the inhibitor, may be provided in particles. Particles as used herein means nano or microparticles (or in some instances larger) which can consist in whole or in part of the inhibitor or the other therapeutic agent(s) as described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero order release, first order release, second order release, delayed release, sustained release, immediate release, and any combination thereof, etc. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules which contain the inhibitor in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described by Sawhney et al in Macromolecules (1993) 26, 581-587, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug there from. “Delayed release” may or may not involve gradual release of drug over an extended period of time, and thus may or may not be “sustained release.”

Use of a long-term sustained release implant may be particularly suitable for treatment of chronic conditions. “Long-term” release, as used herein, means that the implant is constructed and arranged to deliver therapeutic levels of the active ingredient for at least 7 days, and preferably 30-60 days. Long-term sustained release implants are well-known to those of ordinary skill in the art and include some of the release systems described above.

The invention also includes kits. The kit has a container housing an TRPC3 channel inhibitor and optionally additional containers with other therapeutics such as anti-hypertensive or anti-cerebral vasospasm drugs. The kit also includes instructions for administering the component(s) to a subject who has or is at risk of having high blood pressure, hypertension, or vasospasm.

In some aspects of the invention, the kit can include a pharmaceutical preparation vial, a pharmaceutical preparation diluent vial, and inhibitor. The vial containing the diluent for the pharmaceutical preparation is optional. The diluent vial contains a diluent such as physiological saline for diluting what could be a concentrated solution or lyophilized powder of inhibitor. The instructions can include instructions for mixing a particular amount of the diluent with a particular amount of the concentrated pharmaceutical preparation, whereby a final formulation for injection or infusion is prepared. The instructions may include instructions for use in a suppository or other device useful according to the invention. The instructions can include instructions for treating a patient with an effective amount of inhibitor. It also will be understood that the containers containing the preparations, whether the container is a bottle, a vial with a septum, an ampoule with a septum, an infusion bag, and the like, can contain indicia such as conventional markings which change color when the preparation has been autoclaved or otherwise sterilized.

Exemplary TRPC3 channel inhibitors are described above. Other activity inhibitors or antagonists may be identified by those of skill in the art following the guidance described herein.

Libraries of compounds or other putative compounds can be screened to identify other activity inhibitors. Putative compounds can be synthesized from peptides or other biomolecules including but not limited to saccharides, fatty acids, sterols, isoprenoids, purines, pyrimidines, derivatives or structural analogs of the above, or combinations thereof and the like. Phage display libraries and chemical combinatorial libraries can be used to develop and select synthetic compounds which are capable of inhibiting TRPC3 channels. Also envisioned in the invention is the use of compounds made from peptoids, random bio-oligomers (U.S. Pat. No. 5,650,489), benzodiazepines, diversomeres such as dydantoins, benzodiazepines and dipeptides, nonpeptidal peptidomimetics with a beta-D-glucose scaffolding, oligocarbamates or peptidyl phosphonates.

Library technology can be used to identify small molecules, including small peptides, which bind to a TRPC3 channel ligand binding site, or a protein interaction domain of an TRPC3 channel. One advantage of using libraries for antagonist identification is the facile manipulation of millions of different putative candidates of small size in small reaction volumes (i.e., in synthesis and screening reactions). Another advantage of libraries is the ability to synthesize antagonists which might not otherwise be attainable using naturally occurring sources, particularly in the case of non-peptide moieties.

Many if not all of these compounds can be synthesized using recombinant or chemical libraries. A vast array of candidate compounds can be generated from libraries of synthetic or natural compounds. Libraries of natural compounds in the form of bacterial, fungal, plant and animal extracts are available or can readily produced. Natural and synthetically produced libraries and compounds can be readily modified through conventional chemical, physical, and biochemical means. In addition, compounds known to bind to and thereby act as antagonists of calcium channels may be subjected to directed or random chemical modifications such as acylation, alkylation, esterification, amidification, etc. to produce structural analogs which may function similarly or perhaps with greater specificity.

Small molecule combinatorial libraries may also be generated. A combinatorial library of small organic compounds is a collection of closely related analogs that differ from each other in one or more points of diversity and are synthesized by organic techniques using multi-step processes. As an example, analogs of gadolinium chloride or lanthanum chloride can be generated which function as TRPC3 channel inhibitors or antagonists but which don't inhibit other TRPC3 channels. Analogs of these compounds can be synthesized using combinatorial libraries.

Combinatorial libraries include a vast number of small organic compounds. One type of combinatorial library is prepared by means of parallel synthesis methods to produce a compound array. A “compound array” as used herein is a collection of compounds identifiable by their spatial addresses in Cartesian coordinates and arranged such that each compound has a common molecular core and one or more variable structural diversity elements. The compounds in such a compound array are produced in parallel in separate reaction vessels, with each compound identified and tracked by its spatial address. Examples of parallel synthesis mixtures and parallel synthesis methods are provided in PCT published patent application WO95/18972, published Jul. 13, 1995 and U.S. Pat. No. 5,712,171 granted Jan. 27, 1998 and its corresponding PCT published patent application WO96/22529, which are hereby incorporated by reference.

The compounds generated using the recombinant and chemical libraries described herein can be initially screened to identify putative compounds by virtue of their ability to bind to TRPC3 channel. Compounds such as library members can be screened for their ability to bind to TRPC3 channel in vitro using standard binding assays well known to the ordinary artisan and described below. For binding to TRPC3 channel, the TRPC3 channel may be presented in a number of ways including but not limited to cells expressing the TRPC3 channel of interest, an isolated extracellular domain of an TRPC3 channel, a fragment thereof or a fusion protein of the extracellular domain of an TRPC3 channel and another protein such as an immunoglobulin or a GST polypeptide or in a purified (e.g., a recombinantly produced form). For some high throughput screening assays the use of purified forms of an TRPC3 channel, its extracellular domain or a fusion of its extracellular domain with another protein may be preferable. Isolation of binding partners may be performed in solution or in solid state according to well-known methods.

Standard binding assays are well known in the art, and a number of these are suitable in the present invention including ELISA, competition binding assay, sandwich assays, radioreceptor assays using radioactively labeled ligands or substrates of TRPC3 channels (with the binding of the native, radioactively labeled, activator being competed with by the putative antagonist), electrophoretic mobility shift assays, immunoassays, cell-based assays such as two- or three-hybrid screens, etc. The nature of the assay is not essential provided it is sufficiently sensitive to detect binding of a small number of library members.

A variety of other reagents also can be included in the binding mixture. These include reagents such as salts, buffers, neutral proteins (e.g., albumin), detergents, etc. which may be used to facilitate optimal molecular interactions. Such a reagent may also reduce non-specific or background interactions of the reaction components. Other reagents that improve the efficiency of the assay may also be used. The mixture of the foregoing assay materials is incubated under conditions under which the TRPC3 channel normally specifically binds one or more of its activators. The order of addition of components, incubation temperature, time of incubation, and other parameters of the assay may be readily determined. Such experimentation merely involves optimization of the assay parameters, not the fundamental composition of the assay. Incubation temperatures typically are between 4° C. and 40° C. Incubation times preferably are minimized to facilitate rapid, high throughput screening, and typically are between 0.1 and 10 hours. After incubation, the presence or absence of specific binding between the compounds is detected by any convenient method available to the user.

Typically, a plurality of assay mixtures are run in parallel with different agent concentrations to obtain a different response to the various concentrations. One of these concentrations serves as a negative control, i.e., at zero concentration of agent or at a concentration of agent below the limits of assay detection.

Once compounds have been identified which are capable of interacting with TRPC3 channel these compounds can be further screened for their ability to modulate ion flux across these channels. An exemplary assay for measuring the effect of a compound on ion flux is described in the Examples.

As mentioned above, the invention embraces antisense oligonucleotides that selectively bind to a nucleic acid molecules encoding an TRPC3 channel to decrease expression and activity of this protein and subunits thereof.

As used herein, the term “antisense oligonucleotide” or “antisense” describes an oligonucleotide that is an oligoribonucleotide, oligodeoxyribonucleotide, modified oligoribonucleotide, or modified oligodeoxyribonucleotide which hybridizes under physiological conditions to DNA comprising a particular gene or to an mRNA transcript of that gene and, thereby, inhibits the transcription of that gene and/or the translation of that mRNA. The antisense molecules are designed so as to interfere with transcription or translation of a target gene upon hybridization with the target gene or transcript. Antisense oligonucleotides that selectively bind to a nucleic acid molecule encoding an TRPC3 channel are particularly preferred. Those skilled in the art will recognize that the exact length of the antisense oligonucleotide and its degree of complementarity with its target will depend upon the specific target selected, including the sequence of the target and the particular bases which comprise that sequence.

It is preferred that the antisense oligonucleotide be constructed and arranged so as to bind selectively with the target under physiological conditions, i.e., to hybridize substantially more to the target sequence than to any other sequence in the target cell under physiological conditions. Based upon the nucleotide sequences of nucleic acid molecules encoding TRPC3 channel, (e.g., GenBank Accession Nos. NM_{—}003305 Homo sapiens transient receptor potential cation channel, subfamily C, member 3 (TRPC3), mRNA) or upon allelic or homologous genomic and/or cDNA sequences, one of skill in the art can easily choose and synthesize any of a number of appropriate antisense molecules for use in accordance with the present invention. In order to be sufficiently selective and potent for inhibition, such antisense oligonucleotides should comprise at least about 10 and, more preferably, at least about 15 consecutive bases which are complementary to the target, although in certain cases modified oligonucleotides as short as 7 bases in length have been used successfully as antisense oligonucleotides (Wagner et al 1995 Nat Med. 1, 1116-1118). Most preferably, the antisense oligonucleotides comprise a complementary sequence of 20-30 bases. Although oligonucleotides may be chosen which are antisense to any region of the gene or mRNA transcripts, in preferred embodiments the antisense oligonucleotides correspond to N-terminal or 5′ upstream sites such as translation initiation, transcription initiation or promoter sites. In addition, 3′-untranslated regions may be targeted by antisense oligonucleotides. Targeting to mRNA splicing sites has also been used in the art but may be less preferred if alternative mRNA splicing occurs. In addition, the antisense is targeted, preferably, to sites in which mRNA secondary structure is not expected (e.g., Sainio et al 1994 Cell Mol Neurobiol 14, 439-457) and at which proteins are not expected to bind.

In one set of embodiments, the antisense oligonucleotides of the invention may be composed of “natural” deoxyribonucleotides, ribonucleotides, or any combination thereof. That is, the 5′ end of one native nucleotide and the 3′ end of another native nucleotide may be covalently linked, as in natural systems, via a phosphodiester internucleoside linkage. These oligonucleotides may be prepared by art recognized methods which may be carried out manually or by an automated synthesizer. They also may be produced recombinantly by vectors.

In preferred embodiments, however, the antisense oligonucleotides of the invention also may include “modified” oligonucleotides. That is, the oligonucleotides may be modified in a number of ways which do not prevent them from hybridizing to their target but which enhance their stability or targeting or which otherwise enhance their therapeutic effectiveness.

The term “modified oligonucleotide” as used herein describes an oligonucleotide in which (1) at least-two of its nucleotides are covalently linked via a synthetic internucleoside linkage (i.e., a linkage other than a phosphodiester linkage between the 5′ end of one nucleotide and the 3′ end of another nucleotide) and/or (2) a chemical group not normally associated with nucleic acid molecules has been covalently attached to the oligonucleotide. Preferred synthetic internucleoside linkages are phosphorothioates, alkylphosphonates, phosphorodithioates, phosphate esters, alkylphosphonothioates, phosphoramidates, carbamates, carbonates, phosphate triesters, acetamidates, carboxymethyl esters and peptides.

The term “modified oligonucleotide” also encompasses oligonucleotides with a covalently modified base and/or sugar. For example, modified oligonucleotides include oligonucleotides having backbone sugars which are covalently attached to low molecular weight organic groups other than a hydroxyl group at the 3′ position and other than a phosphate group at the 5′ position. Thus modified oligonucleotides may include a 2′-O-alkylated ribose group. In addition, modified oligonucleotides may include sugars such as arabinose instead of ribose.

The present invention, thus, contemplates pharmaceutical preparations containing modified antisense molecules that are complementary to and hybridizable with, under physiological conditions, nucleic acid molecules encoding TRPC3 channel, together with pharmaceutically acceptable carriers. Antisense oligonucleotides may be administered as part of a pharmaceutical composition. In this latter embodiment, it may be preferable that a slow intravenous administration be used. Such a pharmaceutical composition may include the antisense oligonucleotides in combination with any standard physiologically and/or pharmaceutically acceptable carriers which are known in the art. The compositions should be sterile and contain a therapeutically effective amount of the antisense oligonucleotides in a unit of weight or volume suitable for administration to a subject.

The methods of the invention also encompass use of isolated short RNA that directs the sequence-specific degradation of TRPC3 channel mRNA through a process known as RNA interference (RNAi). The process is known to occur in a wide variety of organisms, including embryos of mammals and other vertebrates. It has been demonstrated that dsRNA is processed to RNA segments 21-23 nucleotides (nt) in length, and furthermore, that they mediate RNA interference in the absence of longer dsRNA. Thus, these 21-23 nt fragments are sequence-specific mediators of RNA degradation and are referred to herein as siRNA or RNAi. Methods of the invention encompass the use of these fragments (or recombinantly produced or chemically synthesized oligonucleotides of the same or similar nature) to enable the targeting of TRPC3 channel mRNAs for degradation in mammalian cells useful in the therapeutic applications discussed herein.

The methods for design of the RNA's that mediate RNAi and the methods for transfection of the RNAs into cells and animals is well known in the art and are readily commercially available (Verma et al 2004 J Clin Pharm Ther 28, 395-404; Mello et al 2004 Nature 431, 338-342; Dykxhoorn et al 2003 Nat Rev Mol Cell Biol 4,457-67; Proligo (Hamburg, Germany), Pierce Chemical (part of Perbio Science, Rockford, Ill., USA), Glen Research (Sterling, Va., USA), ChemGenes (Ashland, Mass., USA), and Cruachem (Glasgow, UK)). The RNAs are preferably chemically synthesized using appropriately protected ribonucleoside phosphoramidites and a conventional DNA/RNA synthesizer. Most conveniently, siRNAs are obtained from commercial RNA oligo synthesis suppliers listed herein. In general, RNAs are not too difficult to synthesize and are readily provided in a quality suitable for RNAi. A typical 0.2 μmol-scale RNA synthesis provides about 1 milligram of RNA, which is sufficient for 1000 transfection experiments using a 24-well tissue culture plate format.

The TRPC3 channel cDNA specific siRNA is designed preferably by selecting a sequence that is not within 50-100 bp of the start codon and the termination codon, avoids intron regions, avoids stretches of 4 or more bases such as AAAA, CCCC, avoids regions with GC content <30% or >60%, avoids repeats and low complex sequence, and it avoids single nucleotide polymorphism sites. The TRPC3 channel siRNA may be designed by a search for a 23-nt sequence motif AA(N₁₉). If no suitable sequence is found, then a 23-nt sequence motif NA(N₂₁) may be used with conversion of the 3′ end of the sense siRNA to TT. Alternatively, the TRPC3 channel siRNA can be designed by a search for NAR(N₁₇)YNN. The target sequence may have a GC content of around 50%. The siRNA targeted sequence may be further evaluated using a BLAST homology search to avoid off target effects on other genes or sequences. Negative controls are designed by scrambling targeted siRNA sequences. The control RNA preferably has the same length and nucleotide composition as the siRNA but has at least 4-5 bases mismatched to the siRNA. The RNA molecules of the present invention can comprise a 3′ hydroxyl group. The RNA molecules can be single-stranded or double stranded; such molecules can be blunt ended or comprise overhanging ends (e.g., 5′, 3′) from about 1 to about 6 nucleotides in length (e.g., pyrimidine nucleotides, purine nucleotides). In order to further enhance the stability of the RNA of the present invention, the 3′ overhangs can be stabilized against degradation. The RNA can be stabilized by including purine nucleotides, such as adenosine or guanosine nucleotides. Alternatively, substitution of pyrimidine nucleotides by modified analogues, e.g., substitution of uridine 2 nucleotide 3′ overhangs by 2′-deoxythymidine is tolerated and does not affect the efficiency of RNAi. The absence of a 2′ hydroxyl significantly enhances the nuclease resistance of the overhang in tissue culture medium.

The RNA molecules used in the methods of the present invention can be obtained using a number of techniques known to those of skill in the art. For example, the RNA can be chemically synthesized or recombinantly produced using methods known in the art. Such methods are described in U.S. Published Patent Application Nos. US2002-0086356A1 and US2003-0206884A1 which are hereby incorporated by reference in their entirety.

The methods described herein are used to identify or obtain RNA molecules that are useful as sequence-specific mediators of TRPC3 channel mRNA degradation and, thus, for inhibiting TRPC3 channel receptor activity. Expression of the TRPC3 channel receptor can be inhibited in humans in order to prevent the disease or condition from occurring, limit the extent to which it occurs or reverse it.

The RNA molecules may also be isolated using a number of techniques known to those of skill in the art. For example, gel electrophoresis can be used to separate RNAs from the combination, gel slices comprising the RNA sequences removed and RNAs eluted from the gel slices. Alternatively, non-denaturing methods, such as non-denaturing column chromatography, can be used to isolate the RNA produced. In addition, chromatography (e.g., size exclusion chromatography), glycerol gradient centrifugation, affinity purification with antibody can be used to isolate RNAs.

Any RNA can be used in the methods of the present invention, provided that it has sufficient homology to the TRPC3 channel receptor gene to mediate RNAi. The RNA for use in the present invention can correspond to the entire TRPC3 channel receptor gene or a portion thereof. There is no upper limit on the length of the RNA that can be used. For example, the RNA can range from about 21 base pairs (bp) of the gene to the full length of the gene or more. In one embodiment, the RNA used in the methods of the present invention is about 1000 bp in length. In another embodiment, the RNA is about 500 bp in length. In yet another embodiment, the RNA is about 22 bp in length. In certain embodiments the preferred length of the RNA of the invention is 21 to 23 nucleotides.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

### EXAMPLES

Materials and Methods

Animals and tissues. Twelve- to 16-week old male Sprague-Dawley rats, spontaneously hypertensive rats (SHR), and Wistar-Kyoto rats (WKL) (Charles River Laboratories, St. Constant, Canada) were studied. All animal use procedures were in accordance with institutional guidelines and approved by the institutional Animal Care and Use Committee at the University of Vermont. Rats were euthanized with an injection of pentobarbitone (150 mg/kg ip) followed by exsanguination. The brain was removed and cerebellar and cerebral arteries (between 125 and 225 μm diameter) were dissected free in ice-cold MOPS-buffered saline solution containing (in mM): 3 MOPS, 145 NaCl, 5 KCl, 1 MgSO₄.7H₂O, 2.5 CaCl₂, 1 KH₂PO₄, 0.02 EGTA, 2 pyruvate, 5 glucose and 1% bovine serum albumin (BSA) at pH 7.4. Angiotensin II or a control vehicle (physiological saline solution) was administered for 12 days using a subcutaneous osmotic pump. Blood pressures (MAP) were measured by tailcuff just prior to euthanasia and averaged 93±2 mm Hg in control rats and 146±5 mm Hg in Angiotensin II treated rats.

RT-PCR analysis. RNA was prepared from arteries or isolated smooth muscle cells using the RNeasy kit (Qiagen, Valencia, Calif., USA). 3-5 μL of each first strand cDNA reaction was subsequently placed in a PCR reaction solution (40-45 μL; Applied Biosystems, Branchburg, N.J., USA) containing 1.4 mM MgCl₂, 20 μM forward and reverse primers (Great American Gene Co., Ramona, Calif., USA), 0.25 mM deoxynucleotide-triphosphates, 1× reaction buffer and 2.5 U AmpliTaq Gold DNA polymerase. PCR reactions were hot started (94° C. for 10 min) and then exposed to 35-40 cycles of 94° C. for 60 s, 60° C. for 90 s, and 72° C. for 60 s. Forward and reverse primers specific for TRPC3 were designed using Vector NTI software and were as follows: TRPC3F 5′-CCTGAGCGAAGTCACACTCCCAC-3′ (SEQ ID: 1); TRPC3R 5′-CCACTCTACATCACTGTCATCC-3′ (SEQ ID: 2). Primers yield product sizes of 529 base pairs for TRPC3. All reaction products were resolved on 1% agarose gels.

Western Analysis. Arterial segments were homogenized in lysis buffer (5 minutes, 4° C.) containing (in mM): 40 CAPS, 1 DL-Dithiothreitol (DTT), 10 EDTA, 15 MgCl₂, 115 NaCl, 1 NaOrthovanadate, 1 NaF, 2.5 Urea, and 0.25% deoxychlorate, 10% glycerol, 1% NP-40, 0.2% SDS, and 1:50 mammalian protease inhibitor cocktail (Sigma, St. Louis, Mo., USA). Equal amounts of sample protein were separated on a 10% polyacrylamide gel and transferred to a nitrocellulose membrane. The membranes were exposed to a TRPC3 or TRPC6 polyclonal antibody (anti-rabbit 1:200 dilution, Alomone Labs, Jerusalem, Israel) and a glyceraldehyde dehydrogenase (GAPDH) monoclonal antibody (anti-mouse, 1:1000 dilution, Chemicon Labs, Temecula, Calif., USA). Alexa-Fluor 680® goat anti-rabbit (Molecular Probes, Eugene, Oreg., USA) and IRDyeTM800 anti-mouse (Rockland Immunochemicals, Gilbertsville, Pa., USA) were used to fluorescently label the TRPC3 and GAPDH antibodies respectively. The density of signals specific for the TRPC3 and GAPDH bands in a given lane on a membrane were measured after scanning the membrane with an Odyssey® infared imaging system (Li-COR Biosciences, Lincoln, Nebr., USA). Quantified amounts were normalized to a total protein amount lane on the polyacrylamide gel.

Immunohistochemistry. Freshly isolated arterial segments were fixed for 15 minutes in phosphate buffered saline containing 4% paraformaldehyde and exposed to the primary antibody (1:250 dilution of rabbit Anti-TRPC3; Alomone Labs, Jerusalem, Israel) overnight. Alexa-Fluor 680® goat anti-rabbit (Molecular Probes, Eugene, Oreg., USA) was used to fluorescently label the TRPC3 antibody. The arteries were examined at 40× magnification using a BioRad 1000 laser scanning confocal microscope.

Oligodeoxynucleotide Sequences and Reverse Permeabilization. TRPC6 sense and antisense oligodeoxynucleotides (ODN's) were designed as previously described (Welsh et al 2002 Circ Res 90, 248-250) and were as follows: sense, 5′-CCCTAGCCAGTCTGAACTCC-3′ (SEQ ID: 3) and 5′-GCACACGCAGCCTCTTCAC-3′ (SEQ ID: 4) antisense, 5′-GGAGTTCAGACTGGCTAGGG-3′ (SEQ ID: 5) and 5′-GCACACGCAGCCTCCTTCAC-3′ (SEQ ID: 6). TRPC3 sense and antisense ODN's were designed based on the rat TRPC3 gene (Ohki et al 2000 J Biol Chem 275: 39055-39060) and are as follows: sense, 5′-TATTCCAGTTCATGGTTCTC-3′ (SEQ ID: 7) and 5′-TGTCTGGTCGTGTTGGTCGT-3′ (SEQ ID: 8); anti-sense, 5′-GAGAACCATGAACTGGAATA-3′ (SEQ ID: 9) and 5′-ACGACCAACACGACCAGACA-3′ (SEQ ID: 10). The last five bases on the 5′ and 3′ end were phosphorothioated in order to limit ODN degradation. For some experiments fluoroscein-isothiocyanate was conjugated to the 5′ end to allow for histological assessment of cellular uptake of the ODNs. All ODNs were synthesized and HPLC purified commercially by Qiagen (Alameda, Calif., USA). Sense and antisense ODN's (2 μM) were introduced into the arterial SMs using a reversible permeabilization procedure (Lesh et al 1995 Circ Res 77,220-230)). The arterial segments were then organ cultured for 3 days in D-MEM/F-12 culture media with L-glutamine (2 mM), penicillin (50 units/ml) and streptomycin (50 μg/ml) prior to use.

Patch clamp electrophysiology. Single smooth muscle cells were enzymatically isolated from sense- or antisense-treated cerebral arteries using an isolation solution of the following composition (in mM): 60 NaCl, 58 sodium glutamate, 5.6 KCl, 2 MgCl₂, 10 glucose, 0.1 CaCl₂, 10 HEPES (pH 7.4) with 0.5 mg/ml papain, and 1 mg/ml dithioerythritol added. After 10 minutes, arterial segments were placed in a second isolation solution containing 0.1 mM CaCl₂ and a collagenase type F and hyaluronidase mixture (1 mg/ml each). Trituration was performed with a polished wide-bore pipette and the cells were stored on ice and used the same day. Whole cell ionic currents were measured using the perforated patch method in the presence or absence of 30 μM UTP. Recording electrodes (4-7 MΩ resistance) were pulled from borosilicate glass. Plated cells were voltage clamped and held at −60 mV for 15 minutes prior to experimentation; whole cell currents were monitored while voltage was slowly ramped (−120 to +20 mV, 0.167 mV/ms); currents were not leak subtracted. The bath solution contained (in mM): 120 NaCl, 1.2 MgCl₂, 1.8 CaCl₂, 10 HEPES (pH 7.4), and 10 glucose. The pipette solution contained (in mM): 120 CsCl, 3 MgCl₂, 0.1 EGTA, 10 HEPES (pH 7.2), 10 glucose and 200 μg/ml amphotericin B. A series resistance of approximately 40 MΩ was accepted for all perforated patch clamp experiments. Membrane currents were filtered at 1 kHz, digitized at 5 kHz and stored in a personal computer system for subsequent analysis. pClamp 8.1 and Clampfit 8.1 (Axon Instruments) were used to record and analyze membrane currents. Cell capacitance was measured with the cancellation circuitry of the voltage-clamp amplifier (Axopatch 200A amplifier, Axon Instruments, Sunnyvale, Calif., USA). All current recordings were performed at room temperature (22° C.).

Diameter and membrane potential recordings. Endothelial cell denuded artery segments were mounted on glass pipettes in an arteriograph chamber (Living Systems, Burlington, Vt., USA), pressurized to 20 mmHg (with no flow), and superfused with warm (37° C.), gassed (95% 02/5% CO₂) physiological saline solution (PSS) containing (in mM): 119 NaCl, 4.7 KCl, 24 NaHCO₃, 0.2 KH₂PO₄, 1.1 EDTA, 1.2 MgSO₄, 1.6 CaCl₂, and 10.6 glucose, pH 7.4. In experiments using Ca²⁺-free PSS, CaCl₂ was omitted and 3 mM EGTA and 30 μM diltiazem added. In experiments with diacylglycerol induction, 300 μM of the diacylglycerol analogue OAG (1-oleyol-2-acetyl-sn-glycerol) was added. The endothelium was removed as previously described (Sun et al 1993 Endothelium 1, 115-122). To verify endothelial cell removal the arteries were pressurized to 60 mmHg and allowed to develop myogenic tone prior to exposing the vessels to 1 μM UTP. Absence of a dilation or biphasic constrictor response to 1 μM UTP indicated successful endothelial cell removal (Marelli 2001 Am J Physiol Heart Cir Physiol 281, H1759-H1766; Miyagi et al 1996 Br J Pharmacol 118, 847-856). Arterial diameter or membrane potential (Vm) of sense and anti-sense treated arteries was measured in the absence (control) or presence of UTP (concentration range: 0.1 to 10 μM). Membrane potential was measured by inserting a sharp glass electrode (≈100 MΩ resistance) containing 0.5M KCl into the vessel wall. The criteria for successful vascular smooth muscle cell impalement were: (1) a sharp negative Vm deflection on entry; (2) a stable potential for at least 1 minute after entry; and (3) a sharp positive Vm deflection upon removal. Measurements were made using an electrometer (World Precision Instruments) and the data were recorded via computer using Axotape and Dataq software. Arterial diameter was measured using a video dimension analyzer (IonOptix Corporation; Milton, Mass.).

Ca²⁺ concentration measurement in isolated arteries. Mounted arteries were equilibrated in 1.6 m M PSS for 40 minutes at 37° C. (pH 7.4) prior to loading the vascular smooth muscle cells with the Ca²⁺-sensitive fluorescent dye, FURA-2. To load the cells, the 1.6 mM PSS was gradually replaced over 5 minutes with HEPES solution (25° C., pH 7.4). A total of 40 μL of a premixed 100 μM FURA-2-acetoxymethyl ester solution (100 μg FURA-2, 200 μL Pluronic acid, 800 μL DMSO) was added directly to the HEPES to give a final concentration of 2 μM FURA-2 AM. Loading continued in the dark at 25° C. (pH 7.4) for 40 minutes. At the end of the loading period, the HEPES was replaced with 1.6 mM PSS (35° C., pH 7.4) and 30 minutes was allowed for complete de-esterification of the FURA-2 AM before [Ca²⁺]_{c} measurements were made. FURA-2 fluorescence was measured using a photomultiplier system (IonOptix Corporation; Milton, Mass., USA) in which background-corrected ratios of the 510-nm emission from arteries alternatively excited at 340 and 380 nm were obtained at a sampling rate of 5 Hz.

Chemicals, drugs, and enzymes. Buffer reagents, collagenase type F, hyaluronidase, dithioerythritol and UTP were purchased from Sigma (St Louis, Mo., USA). Papain was obtained from Worthington Biochemical (Lakewood, N.J., USA). Nisoldipine (a gift from Miles Pharmaceuticals, West Haven, Conn., USA) was dissolved in ethyl alcohol to a final solvent concentration of 0.1%. All other compounds were dissolved in the appropriate salt solution.

Statistical analysis. Data are expressed as means±SEM, and n indicates the number of animals. Changes in arterial diameter were measured as a percent constriction calculated as follows:

$$\frac{ϕ ( UTP - NIS ) - ϕ ( UTP )}{ϕ ( initial ) - ϕ ( UTP )} \times 100 , where ϕ = arterial diameter$$

A Student's t-test was used to compare sense to antisense treated experimental groups. In experiments where the treatment group was exposed to more than one concentration of UTP, a two-way repeated measures ANOVA was used. Means were considered significantly different at P≦0.05.

### Example 1

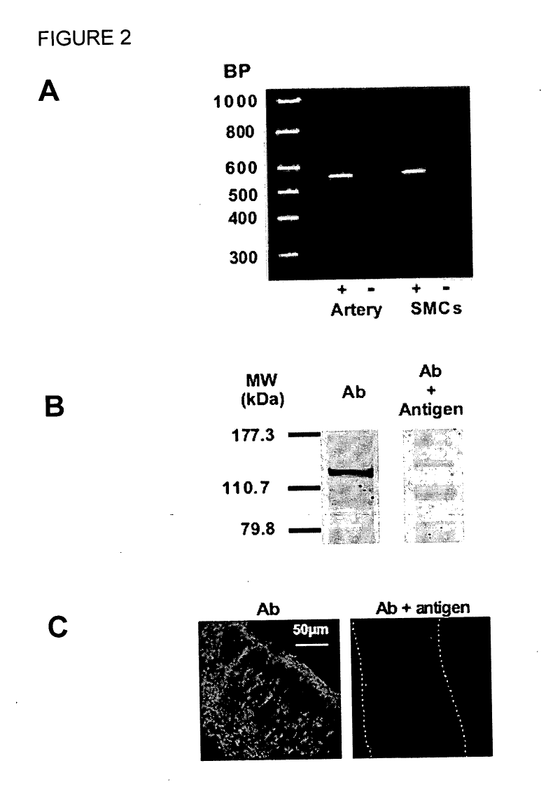

Expression of TRPC3 in rat cerebral arteries. RT-PCR was used to determine if mammalian TRPC3 mRNA transcripts were expressed in cerebral arteries of adult male rats. Messenger RNA for TRPC3 was identified in intact cerebral arteries as well as in smooth muscle cells isolated from these arteries (FIG. 2A). Western analysis of arterial homogenates detected a protein band of approximately 120 kDa that was not detected when the TRPC3 antibody was pre-absorbed with the peptide antigen (FIG. 2B). Immunofluorescent labeling of intact cerebral arteries revealed a circumferential staining pattern for TRPC3 consistent with localization of TRPC3 to the arterial smooth muscle (FIG. 2C).

### Example 2

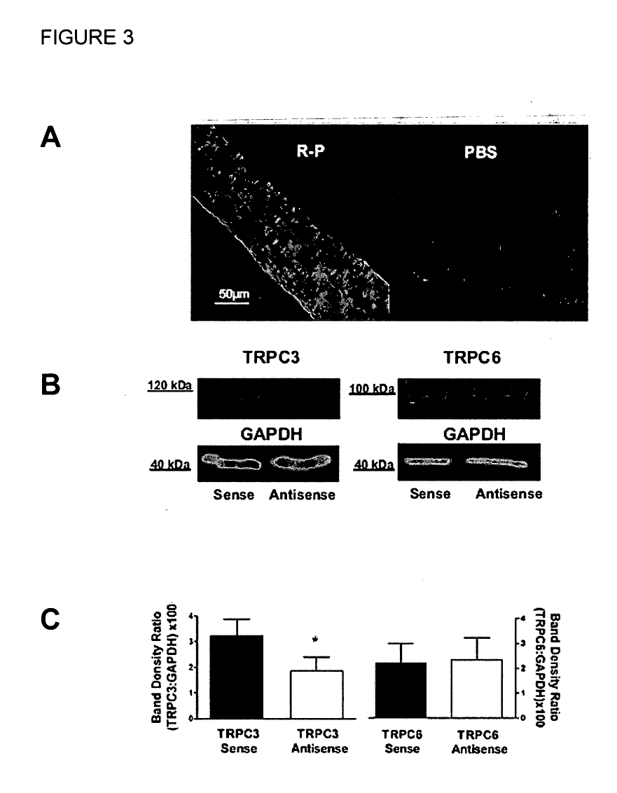

Suppression of TRPC3 expression in cerebral artery. An antisense oligodeoxynucleotide (ODN) approach was employed that has been successfully used in previous studies to reduce TRPC6 channel expression and function (Welsh et al 2002 Circ Res 90, 248-250). In the present Example, it was found that TRPC3 antisense ODNs decreased the expression of TRPC3 when compared to sense-treated arteries. Fluoroscein-labeled ODNs were taken up by cerebral arterial SMC's and the uptake was significantly enhanced by reversibly-permeablizing the arteries (FIG. 3A). Western analysis showed that anti-sense treatment had no effect on the expression of GAPDH but reduced the density of the TRPC3 protein band after 3 days of organ culture (FIG. 3B); the TRPC3 to GAPDH ratio was 42.5±11.4% less in antisense (n=4) when compared to sense (n=4) treated samples (FIG. 3C). Based on previous studies (Muraki et al 1996 Br J Pharmacol 118, 847-856; Sweeney et al 2002 Am J Physiol Lung Cell Mol Physiol 283, L144-L155; Welsh et al 2002 Circ Res 90, 248-250) changes in protein expression of this magnitude are likely to be associated with altered activity of signaling systems that involve the protein of interest. TRPC3 antisense ODNs had no effect on the expression of TRPC6 in cerebral arterial SMCs (FIG. 3B and 3C).

### Example 3

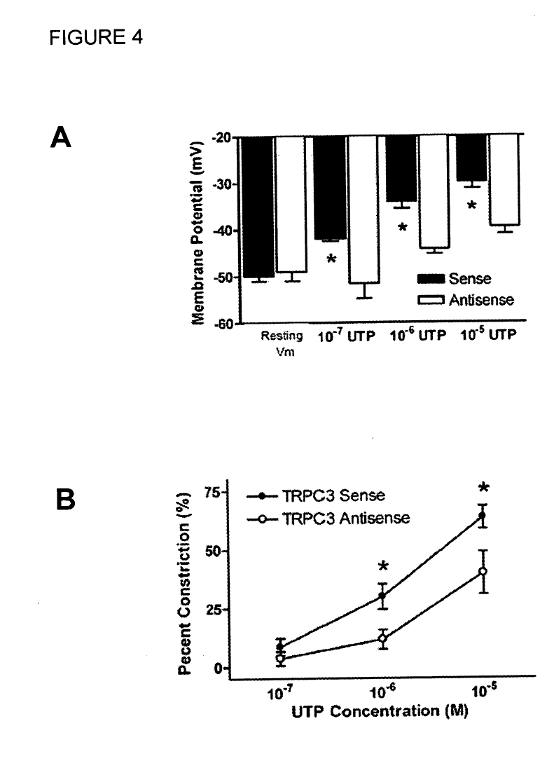

Evidence of a functional role for TRPC3 in rat cerebral arteries. UTP-induced depolarization of SMCs in antisense treated arterial segments was significantly less than in sense-treated arteries (FIG. 4A) at all UTP concentrations tested. In addition to attenuating UTP-induced depolarization of arterial SMCs, suppression of TRPC3 expression also reduced the constrictor responses to UTP over that same concentration range (FIG. 4B). Compared with sense-treated arteries, UTP-induced constrictions of TRPC3 antisense-treated arteries were reduced by approximately 61% in response to 10⁻⁶ M UTP and by 37% in response to 10⁻⁵ M UTP. Antisense ODNs had no generalized inhibitory effect on arterial contractility.

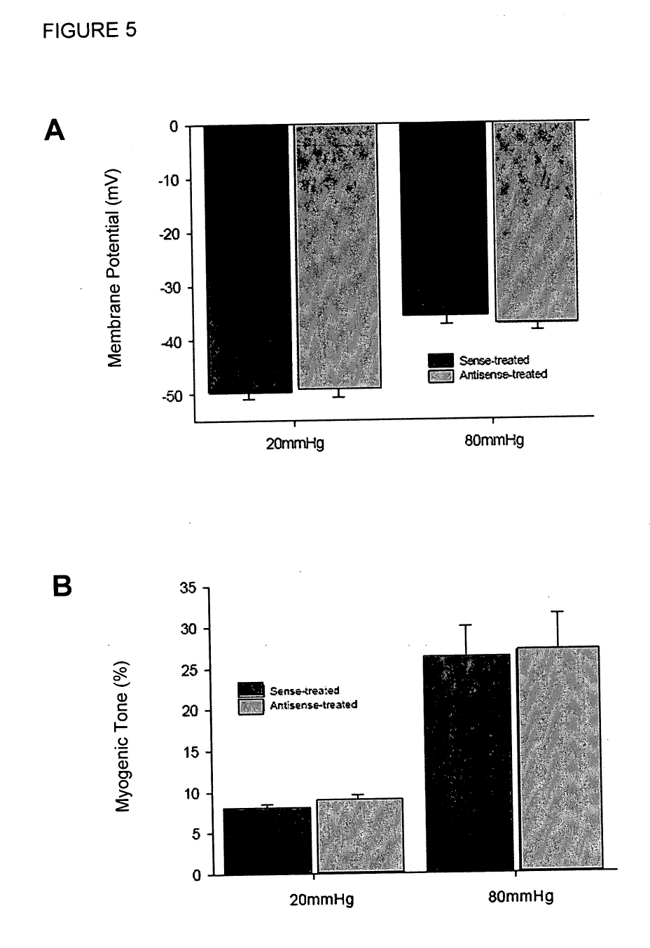

Elevation of extracellular KCl from 5 mM to 60 mM decreased the resting diameter of sense- and anti-sense treated arteries by 58±12% (n=6) and 57±9% (n=7) respectively. Likewise, pressure-induced depolarization and myogenic tone were identical in TRPC3 sense- and antisense-treated arteries. The arterial SMC's depolarized by 14±1 mV in both TRPC3 sense (n=6) and TRPC3 antisense (n=7) arteries, and developed 26±4% (TRPC3 sense) and 27±5% (TRPC3 antisense) myogenic tone when intravascular pressure was increased from 20 to 80 mm Hg (FIG. 5).

### Example 4

Agonist induced depolarization is not mediated by TRPC6 in cerebral artery. It has been shown that TRPC6 is involved in pressure-induced depolarization and myogenic tone in cerebral arteries (Welsh et al 2002 Circ Res 90, 248-250). Interestingly, in the current Example it has been found that exposure to UTP significantly depolarized the SMCs but there was no difference between the TRPC6 sense- and antisense-treated groups. Further, TRPC6 antisense treatment did not affect the magnitude of constriction following exposure to increasing concentrations of UTP indicating that TRPC6 is not involved in UTP-evoked depolarization or constriction of these arteries.

### Example 5

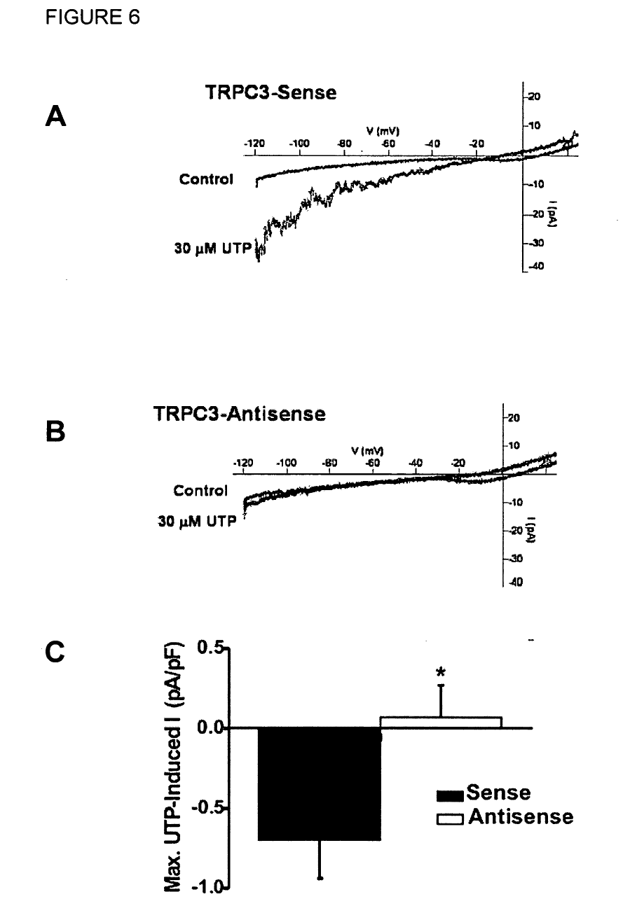

TRPC3 antisense ODNs inhibit a UTP-activated whole-cell current. In further support of the involvement of TRPC3 in the depolarization and constriction induced by UTP, it was found that 30 μM UTP activated a whole cell current in SMCs isolated from TRPC3 sense-treated arteries (8 of 9 cells; voltage ramps from −120 mV to 20 mV) (FIG. 6A). This response to UTP was absent in 3 of 5 SMCs and was greatly suppressed in 2 of 5 SMCs isolated from TRPC3 antisense-treated arteries (FIG. 6B, 6C). These results demonstrate the presence of a UTP-activated current in cerebrovascular smooth muscle cells, and strongly suggest that this current is mediated by TRPC3 channels.

### Example 6

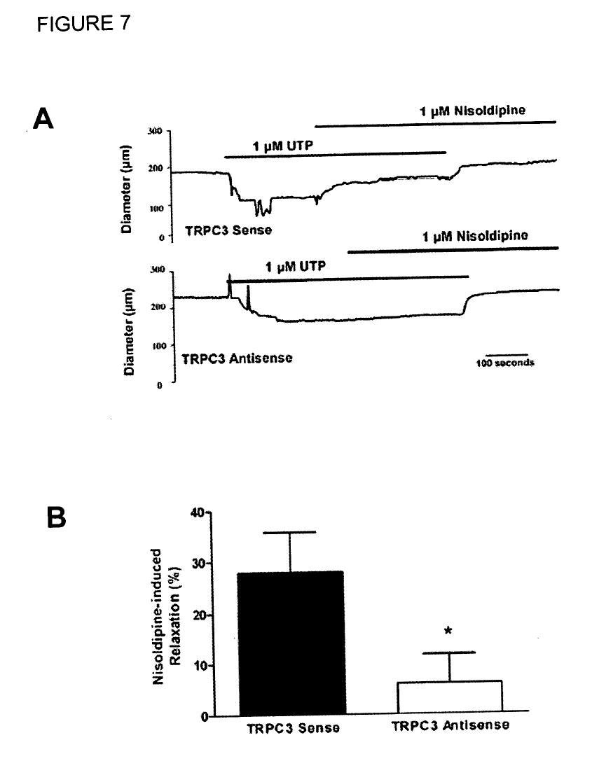

Further evidence of a role for TRPC3 in agonist-evoked depolarization. TRPC3 sense- and antisense-treated arteries were exposed to UTP concentrations sufficient to constrict the arteries by approximately 40%. In the continued presence of UTP, sense and antisense-treated arteries were exposed to 106 M nisoldipine to inhibit voltage-dependent L-type Ca2+ channels. Consistent with the proposal that agonist-induced membrane depolarization contributes to vasoconstriction, we observed that blockade of the L-type Ca2+ channels with nisoldipine partially reversed the contractile response to UTP in sense-treated (FIG. 7A, 7B) but not in antisense-treated arteries. These results indicate that TRPC3 channels mediate UTP-induced depolarization of arterial SMC's, and that the depolarization accounts for a substantial component of the overall vasoconstrictor response.

### Example 7

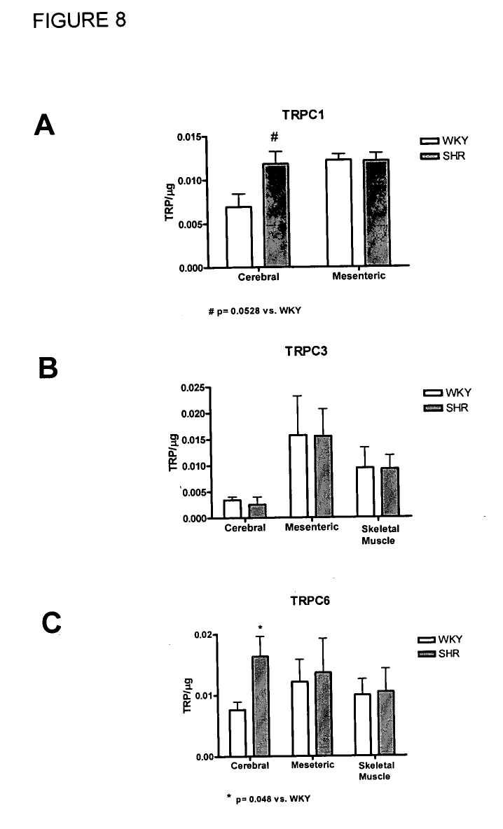

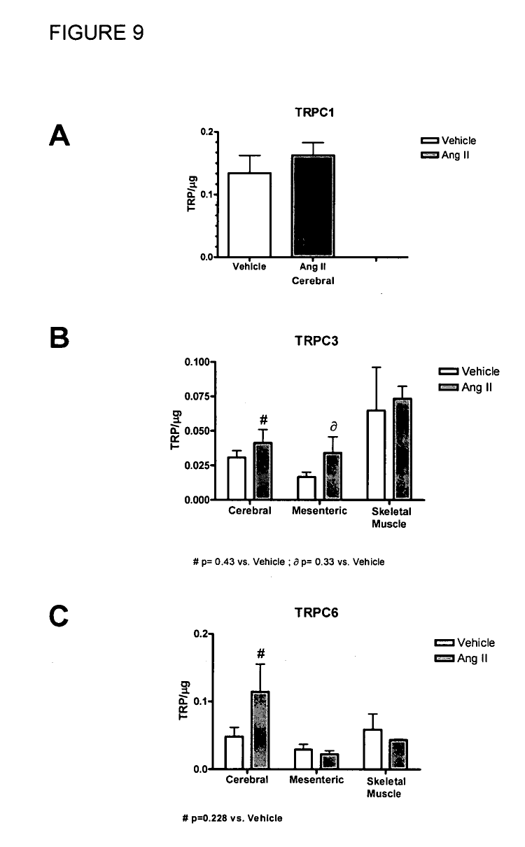

TRPC expression levels in arteries from hypertensive rats. The relative expression of 3 TRPC channel proteins (TRPC1, TRPC3, TRPC6) was measured in resistance arteries (150-200 micrometers lumen diameter) isolated from spontaneously hypertensive rats (SHR), and Wistar-Kyoto rats (WKL) rats, and from Angiotensin II treated (hypertensive) and vehicle treated, (normotensive) Sprague Dawley rats. Arteries from three vascular beds (cerebral, mesenteric, skeletal muscle) were examined. Expression of each of the 3 TRPC proteins was observed in all samples by Western analysis. As shown in FIG. 8C, the SHR showed a significant increase in the expression of TRPC6 in cerebral arteries (p<0.05 vs. WKY). In addition, a trend emerged towards increased expression of TRPC1 in cerebral arteries (p=0.053 vs. WKY)(FIG. 8A). As Shown in FIG. 9B, in Angiotensin-II hypertensive rats, there were trends towards increased expression of TRPC3 in cerebral and mesenteric arteries. In addition, increased expression of TRPC6 when compared to normotensive controls was seen in cerebral arteries (FIG. 9C).

### Example 8

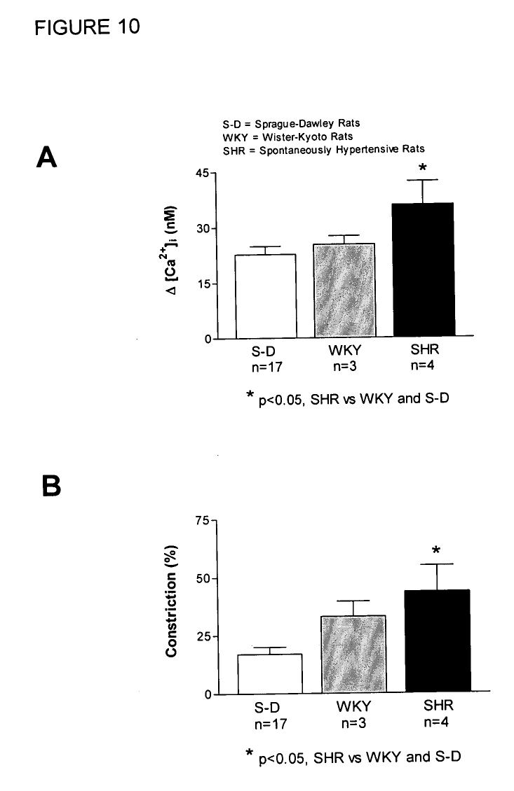

Functional response of intact arteries. Functional responses of isolated, intact cerebral arteries from Sprague Dawley, WKY, and SHR rats were compared (FIG. 10). In these experiments, changes in average (global) intracellular [Ca²⁺] and arterial diameter in response to the diacylglycerol analogue OAG were determined. Diacylglycerol is an agent which is known to activate TRPC3 and TRPC6 channels. The diacylglycerol analogue OAG induced significantly larger increases in calcium concentration and constriction of arteries from SHR rats compared with the normotensive rats. This observation suggests that a vascular TRP channel activated by diacylglycerol is up-regulated in arteries from hypertensive rats. Up-regulation of channel activity could be due to increased channel density and/or increased sensitivity of the channels to diacylglycerol.

### Example 9

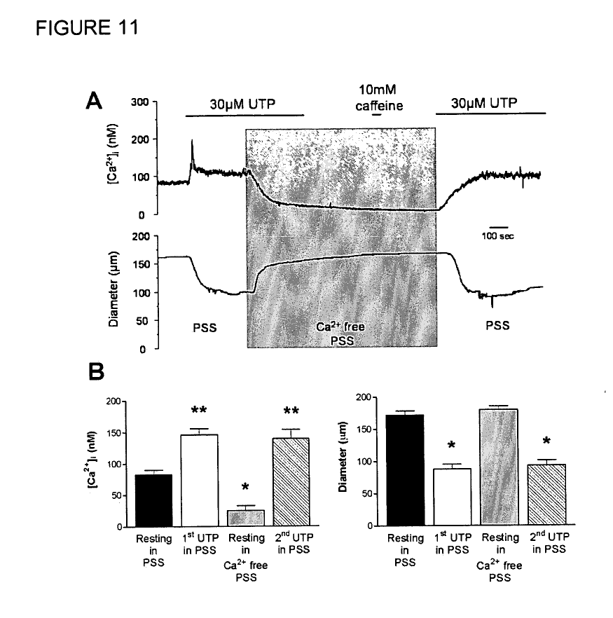

UTP-induced changes in cerebral artery wall [Ca²⁺]_{c} and diameter. FIG. 11A shows representative recordings of wall [Ca²⁺] (upper trace) and lumen diameter (lower trace) of a pressurized cerebral artery loaded with the Ca²⁺ indicator Fura-2. When UTP is added to a PSS (Physiological Saline Solution) superfusate containing Ca²⁺, there is a phasic increase in arterial wall [Ca²⁺]. An initial peak increase in Ca²⁺ (291±45 nM, n=8) declines to a new steady state level that is approximately 2 fold higher (145±10 nM; FIG. 11B) than observed in the absence (71±7 nM; FIG. 11B) of UTP. Corresponding to the increased arterial wall [Ca²⁺] is a sustained 49±4% decrease in lumen diameter (FIG. 11B). Removal of extracellular Ca²⁺ by superfusing the artery with a Ca²⁺ free PSS caused arterial wall [Ca²⁺] to decline below the resting level and the vessel to completely relax despite the continued presence of UTP in the superfusate (FIG. 11A). Returning extracellular Ca²⁺ to the superfusate in the presence of UTP resulted in an increase in arterial wall Ca²⁺ (139±15 nM) and a decrease in lumen diameter (48±5%) of the vessel to steady-state values that were similar to the first UTP exposure.

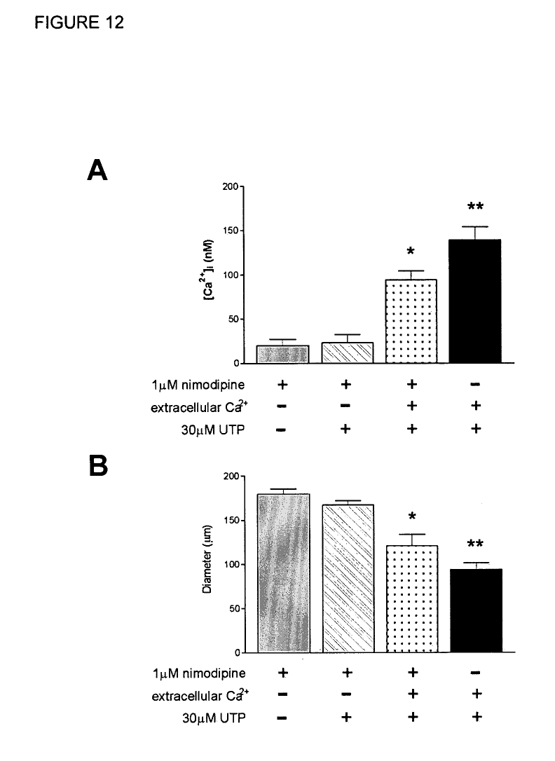

It is unlikely that Ca²⁺ release from intracellular Ca²⁺ stores contributes to the increase in wall Ca²⁺ and vasoconstriction that follows the re-introduction of extracellular Ca²⁺ in the presence of UTP. Absent from this Ca²⁺ response was the transient peak increase (FIG. 11A) that likely occurs as a result of Ca²⁺ release from sarcoplasmic reticulum (SR). Also, arteries superfused with PSS containing Ca²⁺ contract transiently when superfused with 10 mM caffeine (peak [Ca²⁺]_{c} 227±42 nM; % constriction 31±6%) to stimulate SR Ca²⁺ release. In the absence of extracellular Ca²⁺ neither UTP (FIG. 11 and FIG. 12) nor caffeine (FIG. 11A) affected arterial wall Ca²⁺ or vessel diameter indicating that under these experimental conditions the Ca²⁺ stores were likely depleted. Additional experiments (n=3) indicated that in the presence of 2 μM thapsigargin to block Ca²⁺ uptake by the SR, arterial wall Ca²⁺ still increased to 116±29 nM and diameter decreased by 39±6% when extracellular Ca²⁺ was reintroduced in the presence of UTP. Thus, under the experimental conditions used in this study, Ca²⁺ influx across the sarcolemmal membrane and not Ca²⁺ release from the SR is responsible for the increase in arterial wall Ca²⁺ and vasoconstriction that result when extracellular Ca²⁺ is returned to a Ca²⁺ free superfusate in the presence of UTP.

### Example 10

UTP-induced extracellular Ca²⁺ influx pathways. Inhibition of voltage-sensitive L-type Ca²⁺ channels with 1 μM nimodipine completely prevented 60 mM KCL from increasing arterial wall Ca²⁺ or decreasing arterial diameter. The same concentration of nimodipine significantly attenuated, but did not prevent, the UTP-induced increase in arterial wall Ca²⁺ (94±10 nM vs. 139±15 nM) and vasoconstriction (29±3% vs. 48±5%) when extracellular Ca²⁺ was re-introduced to the superfusate in the presence of UTP (FIG. 12). This effect could not be enhanced by using higher concentrations of nimodipine, and similar levels of inhibition were achieved with 1 μM nisoldipine or 30 μM diltiazem to inhibit L-type Ca²⁺ channels. Thus, only about 30% of the extracellular Ca²⁺ influx occurring with the re-admission of Ca²⁺ to the superfusate in the presence of UTP enters the smooth muscle through the L-type Ca²⁺ channel.

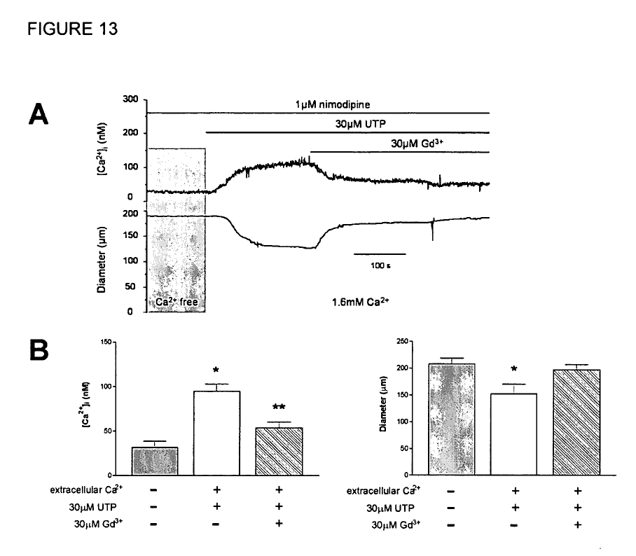

Inhibition of non-selective cation channels with 30 μM Gd³⁺ reverses the increase in arterial wall Ca²⁺ and vasoconstriction that results when extracellular Ca²⁺ is returned to the superfusate in the presence of UTP (FIGS. 13A and B). If the artery was exposed to Gd³⁺ prior to the re-introduction of extracellular Ca²⁺ to the superfusate, then arterial wall Ca²⁺ did not increase nor did vasoconstriction occur in response to UTP.

### Example 11

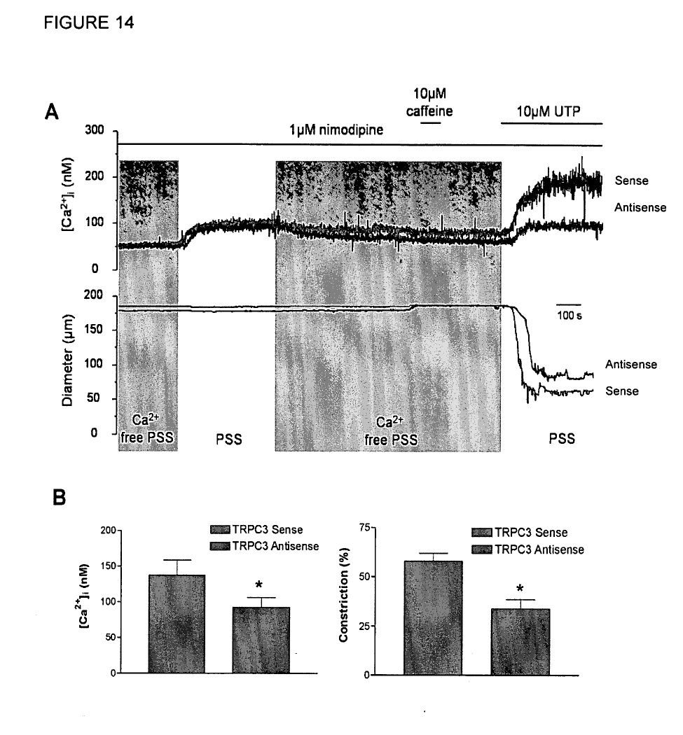

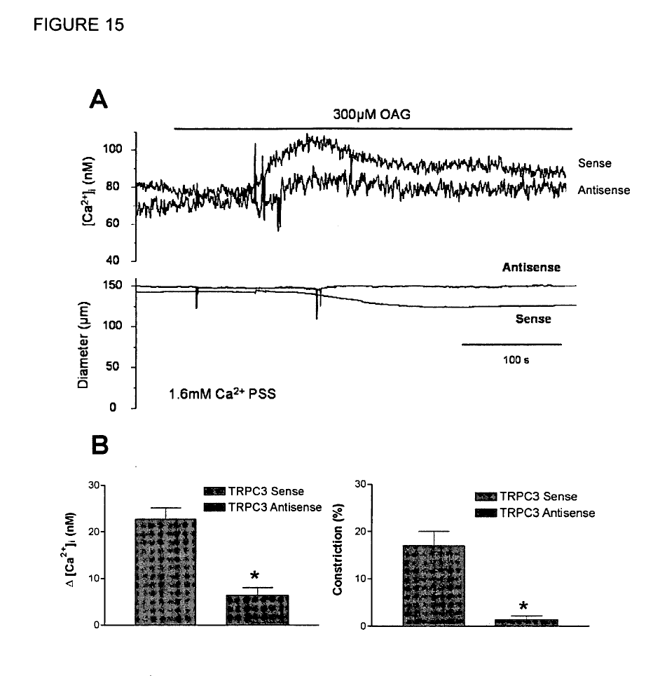

Ca²⁺ influx through TRPC3 channels. FIG. 14 shows arterial wall Ca²⁺ and lumen diameter values for arteries treated with TRPC3 sense or antisense ODNs. Antisense suppression of TRPC3 channel expression significantly reduced the change in arterial wall Ca²⁺ (100±22 nM, sense vs. 50±12 nM, antisense) and lumen diameter (−128±8 μm, sense vs. −85±17 μm, antisense) that occurred when Ca²⁺ was returned to the superfusate in the presence of UTP and nimodipine (FIG. 14). PLC-coupled receptor activation of TRPC3 channels is likely mediated by the second messenger DAG or a downstream product. The data in FIG. 15 show that 300 μM OAG (a membrane permeant analog of DAG known to activate TRPC channels) increases arterial wall Ca²⁺ by 23÷2 nM and 6±2 nM in TRPC3 sense- and antisense-treated arteries respectively. The increase in arterial wall Ca²⁺ causes TRPC3 sense-treated arteries to constrict (17±3%) where as TRPC3 antisense-treated arteries do not respond (1±1%).

The present study demonstrates that TRPC3 channels contribute to UTP-induced constriction of cerebral arterial SMCs in two ways—by mediating smooth muscle cell depolarization, which enhances Ca²⁺ entry through L-type Ca²⁺ channels, and as a direct Ca²⁺ influx pathway per se. Several observations support this conclusion. First, TRPC3 mRNA and protein were expressed in SMCs and cerebral arteries of adult rats (FIG. 2). Second, suppression of TRPC3 channel expression significantly attenuated UTP-induced depolarization and constriction (FIG. 4). Third, suppression of TRPC3 channel expression nearly eliminated UTP-induced whole-cell currents in isolated SMC's (FIG. 5). Fourth, inhibition of voltage-dependent L-type Ca²⁺ channels partially reversed UTP-induced vasoconstriction when TRPC3 channels were present but not when their expression was suppressed. Fifth, under conditions where Ca⁺² entry through L-type Ca²⁺ channels is blocked, UTP continues to induce substantial Ca²⁺ entry (FIG. 12) that is blocked by Gd⁺³, a TRPC channel inhibitor (FIG. 13), and greatly reduced when the expression of TRPC3 channels is suppressed (FIG. 14).

The Examples demonstrate for the first time that TRPC3 channels are involved in receptor-mediated vasoconstriction. The Examples show that antisense suppression of TRPC3 decreased UTP-induced depolarization of cerebral artery SMCs (FIGS. 3 and 4) whereas antisense suppression of TRPC6 was without effect. This indicates that TRPC6 channels are not involved in pyrimidine receptor-mediated response in these cells. Also, suppression of TRPC3 had no effect on pressure-induced depolarization or the development of myogenic tone by cerebral artery SMCs. Suppression of TRPC6 however, does decrease pressure-induced depolarization of cerebral artery SMC's (Welsh et al 2002 Circ Res 90, 248-250). Together these findings demonstrate that different excitatory stimuli, in this case UTP and pressure, are coupled to distinct populations of TRPC channels in cerebral arterial SMCs. Similarly, differences in coupling of specific receptor types (e.g., purinergic vs. adrenergic) to individual TRPC channel isoforms may occur in different arterial SMC types (e.g., cerebral vs. systemic, conduit vs. resistance) or in arterial compared to venous SMCs. Such differences might account for the absence of a UTP-induced depolarizing current mediated by TRPC6 in cerebral arterial SMCs, when a TRPC6-mediated depolarizing current is clearly present in rabbit portal vein SMCs exposed to an α₁-adrenergic agonist (Inoue et al 2001 Circ Res 88, 325-332).

Depolarization and activation of L-type calcium channels in vascular smooth muscle cells is a well-established mechanism of vasoconstriction (Nelson et al 1990 Am J Physiol Heart Circ Physiol 259, C3-C18). Micromolar concentrations of UTP depolarize arterial SMC's by approximately 20 mV (FIG. 4; Luykenaar et al 2004 Am J Physiol Heart Circ Physiol 286, H1088-H1100; Welsh and Brayden 2001 Am J Physiol Heart Circ Physiol 280, H2545-H2553), which is sufficient to open L-type Ca²⁺ channels to permit extracellular Ca²⁺ influx and constriction (FIG. 7; Knot and Nelson 1998 J Physiol (London) 508, 199-209). UTP also activates a TRPC3 mediated inwardly rectifying current in isolated SMC (FIG. 5). Thus, TRPC3 channels are primary mediators of UTP-induced depolarization of cerebral artery smooth muscle.

TRPC3 channels are, thus, channels that contribute to the ion fluxes controlling arterial diameter. TRPC3 channels are distinctly involved in UTP-induced depolarization, Ca²⁺ entry, and constriction of arterial smooth muscle whereas TRPC6 channels contribute to the arterial myogenic response. The unique and differential activation of these ion channels by various excitatory stimuli has important implications concerning the development of therapeutic strategies targeted to specific vascular SMC constrictor mechanisms in vascular disease states.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

What is claimed is:

1. A method for treating hypertension, comprising:
administering to a subject in need thereof an effective amount of a TRPC3 channel inhibitor for treating hypertension.

2. The method of claim 1, wherein the TRPC3 channel inhibitor is an activity inhibitor.

3. The method of claim 2, wherein the activity inhibitor is a small molecule.

4. The method of claim 1, wherein the TRPC3 channel inhibitor is an expression inhibitor.

5. The method of claim 4, where the expression inhibitor is an antisense or siRNA molecule.

6. The method of claim 1, wherein the TRPC3 channel inhibitor is administered orally.

7. The method of claim 1, wherein the TRPC3 channel inhibitor is administered intravenously.

8. The method of claim 1, wherein the TRPC3 channel inhibitor is administered by intra-arterial route.

9. A composition, comprising:
a TRPC3 channel inhibitor and an anti-hypertensive drug formulated with a pharmaceutically-acceptable carrier.

10. The composition of claim 9, wherein the TRPC3 channel inhibitor is an activity inhibitor.

11. The composition of claim 9, wherein the TRPC3 channel inhibitor is an expression inhibitor.

12. The composition of claim 11, wherein the expression inhibitor is an antisense or siRNA molecule.

13. The composition of claim 9, wherein the composition is formulated for oral administration.

14. The composition of claim 9, wherein the composition is formulated for intravenous administration.

15. A method for reducing blood pressure in a subject, comprising:
administering to a subject in need thereof an effective amount of a TRPC3 channel inhibitor for reducing blood pressure in the subject.

16. The method of claim 15, wherein the TRPC3 channel inhibitor is an activity inhibitor.

17. The method of claim 16, wherein the activity inhibitor is a small molecule.

18. The method of claim 15, wherein the TRPC3 channel inhibitor is an expression inhibitor.

19. The method of claim 18, where the expression inhibitor is an antisense or siRNA molecule.

20. The method of claim 15, wherein the subject has or is at risk of developing a vasospasm.

21. The method of claim 20, wherein the vasospasm is a cerebral vasospasm.

22. The method of claim 20, wherein the vasospasm is a coronary artery vasospasm.

23. The method of claim 20, wherein the vasospasm is associated with vascular surgery.

24. The method of claim 20, wherein the vasospasm is peripheral vascular disease.

25. A kit comprising:
a container housing an TRPC3 channel inhibitor and instructions for administering the TRPC3 channel inhibitor to a subject in order to lower the blood pressure of the subject.

26. The kit of claim 25, wherein the subject has hypertension.


SEQUENCE LISTING (10 sequences)

SEQ ID NO: 1 - DNA, 23 bp, artificial sequence
  synthetic oligonucleotide
cctgagcgaagtcacactcccac

SEQ ID NO: 2 - DNA, 22 bp, artificial sequence
  synthetic oligonucleotide
ccactctacatcactgtcatcc

SEQ ID NO: 3 - DNA, 20 bp, artificial sequence
  synthetic oligonucleotide
ccctagccagtctgaactcc

SEQ ID NO: 4 - DNA, 19 bp, artificial sequence
  synthetic oligonucleotide
gcacacgcagcctcttcac

SEQ ID NO: 5 - DNA, 20 bp, artificial sequence
  synthetic oligonucleotide
ggagttcagactggctaggg

SEQ ID NO: 6 - DNA, 20 bp, artificial sequence
  synthetic oligonucleotide
gcacacgcagcctccttcac

SEQ ID NO: 7 - DNA, 20 bp, artificial sequence
  synthetic oligonucleotide
tattccagttcatggttctc

SEQ ID NO: 8 - DNA, 20 bp, artificial sequence
  synthetic oligonucleotide
tgtctggtcgtgttggtcgt

SEQ ID NO: 9 - DNA, 20 bp, Artificial Sequence
  synthetic oligonucleotide
gagaaccatgaactggaata

SEQ ID NO: 10 - DNA, 20 bp, artificial sequence
  synthetic oligonucleotide
acgaccaacacgaccagaca